(19) United States
(12) Patent Application Publication
(10) Pub. No.: US 2026/0216155 A1
(43) Pub. Date: Jul. 30, 2026

(54) Chemotherapeutic Microparticle Compositions

(71) Applicant: eSpraye, Inc., San Francisco, CA (US)

(72) Inventors: Aditya Das, Foster City, CA (US); John Patton, San Francisco, CA (US); Daniel Green, San Francisco, CA (US); Danforth P. Miller, San Carlos, CA (US)

(73) Assignee: eSpraye, Inc., San Francisco, CA (US)

(21) Appl. No.: 19/461,283

(22) Filed: Jan. 27, 2026

Related U.S. Application Data
(60) Provisional application No. 63/750,210, filed on Jan. 27, 2025.

(51) Int. Cl. A61K 31/4412; A61K 9/00; A61K 31/085; A61K 31/496; A61K 47/02; A61K 47/24

(52) U.S. Cl. CPC A61K 31/4412; A61K 9/0075; A61K 31/085; A61K 31/496; A61K 47/02; A61K 47/24

(57) ABSTRACT

This disclosure provides for compositions and methods for treating incipient or moderate pulmonary fibrosis and idiopathic pulmonary fibrosis (collectively pulmonary fibrosis or “PF”) in patients in need thereof. Provided is a dry powder composition comprising an effective amount of a) pirfenidone; and b) nintedanib or a pharmaceutically acceptable salt thereof, wherein the composition is suitable for direct pulmonary delivery. Also contemplated are compositions for direct pulmonary delivery comprising 1 to 5 active pharmaceutical ingredients.

### CROSS REFERENCE TO RELATED APPLICATIONS

This application claims the benefit of U.S. Provisional application 63/750,210, filed on Jan. 27, 2025, the contents of which are incorporated by reference in their entirety for all purposes.

### INCORPORATION BY REFERENCE

All publications, patents, and patent applications cited herein are incorporated by reference to the same extent as if each individual publication, patent, or patent application were specifically and individually indicated to be incorporated by reference. In the event of a conflict between a term herein and a term in an incorporated reference, the term herein controls.

### FIELD

This disclosure provides for compositions and methods for treating incipient or moderate a) pulmonary fibrosis, b) progressive pulmonary fibrosis, and c) idiopathic pulmonary fibrosis (collectively pulmonary fibrosis or “PF”) in patients in need thereof. The methods described herein involve the delivery of a composition suitable for inhalation directly into the deep pulmonary bed (e.g., via a nebulizer, a pressurized metered dose inhaler, or a dry powder inhaler) to inhibit or slow the further development of PF, thereby maintaining or improving the quality of life and delaying end-of-life events for patients with PF. Left untreated, this disease progressively decreases the ability of the lungs to absorb oxygen into the blood, leading to shortness of breath or periods of shortness of breath, progressive dyspnea, and a dry, nonproductive cough in the patient, all of which are characteristic of this disease.

### BACKGROUND

Pulmonary fibrosis (PF) is a disease characterized by fibrotic changes in the lungs, often referred to as scarring. The etiology of certain types of pulmonary fibrosis is known, whereas that of idiopathic pulmonary fibrosis is unknown. Regardless, PF is a chronic lung disease characterized by increasing inflammatory infiltrates and pathological excess in collagen deposition. PF often presents in patients as a nonproductive cough and exertional dyspnea. The clinical course of PF is chronic, and the alteration in the structure and functionality of the lungs ultimately leads to death. PF carries a poor prognosis, with a median survival of 3.8 years among newly diagnosed adults 65 years of age or older in the United States. Despite significant improvements in the diagnosis and treatment of PF, the clinical disease prognosis has not improved.

The underlying pathology of the disease is characterized by ongoing fibrosis that includes the deep pulmonary tract containing the alveoli, leading to blockage of inhaled air from interfacing with the alveoli. The further the disease progresses, the greater the reduction in oxygen absorption, leading to progressive dyspnea. Ultimately, a patient's reduced ability to obtain adequate levels of oxygen due to PF leads to comorbidities such as pulmonary hypertension, heart failure, coronary artery disease, cardiac arrhythmias, and other cardiac manifestations, which, whether alone or in combination, contribute to end-of-life for such patients. To date, there is no cure for PF.

The current treatment for PF typically includes the oral administration of drugs such as pirfenidone and/or nintedanib, both of which are considered anti-fibrotic agents. The literature reports that there is neither evidence from prospective data nor a guideline recommendation on which drug should be preferred over the other. In addition, the intolerably high oral doses described below often preclude patients from adhering to the prescribed therapies. Accordingly, the choice of drug in treating PF is made on a case-by-case basis, relying heavily on the patient's comorbidities and other gastrointestinal side effects from oral dosing. See, e.g., Marijic et al., “Pirfenidone vs. nintedanib in patients with idiopathic pulmonary fibrosis: a retrospective cohort study,” Respir Res. 2021 Oct. 19; 22(1):268. doi: 10.1186/s12931-021-01857-y. PMID: 34666765; PMCID: PMC8527681.

Each of these drugs exhibits a different underlying mechanism of action. For example, pirfenidone reduces further fibrotic progression by inhibiting the production of growth factors and procollagens I and II that promote fibrotic growth, while also exhibiting some anti-inflammatory properties, which is part of the underlying etiology. Ruwanpura, et al., Pirfenidone: Molecular Mechanism and Potential Clinical Applications in Lung Disease. Am J Respir Cell Mol Biol., 2020 April; 62(4): 413-422. Nintedanib binds to tyrosine kinase receptors and is believed to inhibit neoangiogenesis required for fibrotic development. Gole and Bankhole, Nintedanib [Updated 2024 Aug. 17]. In: StatPearls [Internet]. Treasure Island (FL): StatPearls Publishing; 2025 January-. Available from: https://www.ncbi.nlm.nih.gov/books/NBK585049/.

Both drugs are orally delivered, albeit in significantly high but different dosages. The oral bioavailability of pirfenidone is ~46%, and that of nintedanib is ~4% following first-pass metabolism, but the actual translocation to the alveolar epithelium from systemic blood flow is another major hurdle, which is nonquantifiable except for macroscopic endpoints, such as improved Forced Vital Capacity (FVC) and a 6-minute walk distance. For example, the approved oral dosages for pirfenidone start with an initial daily dose of 801 mg/day, taken as two or three capsules. This is followed by a daily maintenance dosage, after day 15, of 2,403 mg per day. Nintedanib is administered at 150 mg twice a day but has only about 4% bioavailability due, at least in part, to its low water solubility at pH values representative of the intestinal lumen, approximately 12 μg/mL at pH 6.8 and approximately 5 μg/mL at pH 7.4.

Both drugs, when administered orally, have significant side effects that limit their use. Pirfenidone, for example, can cause kidney and liver impairment as well as severe gastrointestinal and photosensitivity intolerance. Likewise, nintedanib can also cause severe gastrointestinal problems, including nausea, diarrhea, severe stomach pain, and weight loss, to name a few. As such, a good number of patients have chosen either not to undergo pharmacotherapy or to cease treatment with either or both drugs due to such side effects. Gulati S, Luckhardt T R. Updated Evaluation of the Safety, Efficacy and Tolerability of Pirfenidone in the Treatment of Idiopathic Pulmonary Fibrosis. Drug Healthc. Patient Saf. 2020 May 7; 12:85-94. doi: 10.2147/DHPS.S224007. PMID: 32440226; PMCID: PMC7213901; and Glaspole, I., Bonella, F., Bargagli, E. et al. Efficacy and safety of nintedanib in patients with idiopathic pulmonary fibrosis who are elderly or have comorbidities. Respir Res 22, 125 (2021). https://doi.org/10.1186/s12931-021-01695-y.

Accordingly, there is an ongoing unmet need to find a more effective and tolerated pharmacotherapeutic approach to treating PF and preventing or slowing the progression of PF, especially for delivering a therapeutic drug regimen to treat PF. There is also an ongoing need to bypass oral delivery when such a delivery produces serious adverse side effects. Ideally, alternative delivery protocols should enhance the desired therapy, and/or reduce the amount of drug necessary to achieve a therapeutic result, and/or lower the overall systemic uptake to the patient, and thus fewer side effects.

### SUMMARY

In some embodiments, this disclosure provides for dry powder compositions for treating PF. In some embodiments, the dry powder composition is suitable for direct pulmonary delivery, wherein said dry powder is characterized, in part, by being moisture resistant and room temperature stable. Direct pulmonary delivery is used synonymously with oral inhalation.

In some embodiments, the dry powder compositions described herein comprise an effective amount of either one or both of a) pirfenidone; and b) nintedanib (“actives”). In addition, the drug powder also comprises inactive agents such as c) a lecithin component (e.g., a di-fatty acid phosphatidyl choline), d) a divalent cation (e.g., calcium chloride), and e) up to about 6 percent by weight water.

It is contemplated that in some embodiments of the dry powder compositions, an effective amount of pirfenidone can be substituted with an effective amount of deupirfenidone.

It is contemplated that in some embodiments, the dry powder composition further comprises f) an effective amount of treprostinil or treprostinil palmitil.

It is contemplated that in some embodiments, the dry powder composition further comprises an effective amount of admilparant, buloxibutid, nerandomilast, tadalegib, or vismodegib.

In some embodiments, the effective amount of the active or actives used in the dry powder is/are uniformly dispersed therein.

In some embodiments, the low density of the dry powder composition delivered from an inhalation dispenser into the airways of the lung allows for a major portion of said composition to be deposited in the alveoli of the lungs and the adjacent tissue.

In some embodiments, the dry powder composition comprises particles (e.g., a plurality of particles) having a stable ultra-low density configuration which allows for entrainment in laminar airflow upon slow and deep inhalation of the particles. Such a stable ultra-low density is provided by a bulk density which typically ranges from about 0.03 g/cc to about 0.30 g/cc or about 0.04 g/cc to about 0.11 g/cc (and any 0.1 g/cc value in between those ranges).

In one embodiment, the two drugs, or three drugs if in a contemplated further combination with treprostinil or treprostinil palmitil, are uniformly dispersed in the particles in a ratio proportional to the amount of each used in the composition on a weight basis in an ultra-low density bulk powder. Such a drug-containing low-density bulk powder can be produced by spray drying. An exemplary bulk powder can have a low density, such as about 0.03 g/cc to about 0.30 g/cc, 0.04 to about 0.11 g/cc, 0.06 to about 0.08 g/cc, as well as any 0.01 value between 0.03 g/cc to 0.15 g/cc. The two (or contemplated three actives) active drugs are coated with the di-fatty acid phosphatidyl choline matrix at the ratios used in the spray-drying feedstock, and when the feedstock is spray-dried can produce a dry powder composition suitable for filling into vessels, such as capsules (size 3). The vessel holding the spray-dried powder can be inserted, for instance, into a Dry Powder Inhaler which, when used, will puncture the vessel (e.g., capsule, container, pod, casing, or blister package) followed by subsequent inhalation of the released powder released from the punctured vessel (e.g., a size 3 capsule). When not using a low resistance device, slow and deep inhalation at a flow rate of about 25 to 35 liters per minute (LPM) can be used and result in most of the drug actives in the combination or triple drug therapy (or triple drug combination) being delivered to the lungs.

In some embodiments, the relative weight ratio of pirfenidone to nintedanib in the dry powder compositions disclosed herein is from about 8:1 to about 200:1, from about 8:1 to about 100:1, from about 8:1 to about 80:1, from about 10:1 to about 70:1, from about 12:1 to about 60:1, from about 15:1 to about 50:1, from about 15:1 to about 40:1, from about 20:1 about 40:1, and from about 20:1 to about 30:1. In some embodiments, the relative weight ratio of pirfenidone to nintedanib in the dry powder compositions disclosed herein is from about 8.5:1 to about 30:1; in other embodiments, the ratio of pirfenidone to nintedanib in the dry powder compositions disclosed herein is from about 9:1 to about 30:1 and preferably 25:1. These ratios increase the amount of pirfenidone relative to nintedanib.

It is contemplated that direct delivery of these drugs and drug combinations to the deep pulmonary epithelium at or proximate to the alveoli will reduce the amount of each drug required to achieve a therapeutic result by targeted delivery to the healthy alveolar cells and close, if not direct targeting of the fibrotic cells. It is contemplated that in either case, absorption through the gastrointestinal tract is avoided, as are the multiple side effects associated with the very high systemic exposures from the oral products.

In some embodiments, the aggregate amount of pirfenidone delivered to a patient's lungs using the dry powder compositions described herein is from about 10 mg to 100 mg per day, or about 20 mg to 60 mg per day. Such dosing significantly reduces the oral dose burden (about 500 mg to about 2400 mg daily) and associated side effects. It is contemplated that the direct delivery of pirfenidone to the pulmonary tract of the patient improves the overall absorption either directly into fibrotic cells, thereby reducing the amount of drug required to achieve an efficacious result.

In some embodiments, the aggregate amount of nintedanib delivered to a patient using the dry powder compositions described herein is from about 0.1 mg to about 8.0 mg per day, about 0.1 mg to about 4.0 mg per day, about 0.1 mg to about 2.0 mg per day, or 0.5 mg to about 2.0 mg per day, or about 1.0 mg to 2.0 mg per day. These doses are significantly reduced compared to the oral dose burden (about 100 mg to about 300 mg per day). Reductions in the amount of a drug administered to a patient that are about 20 to 50 times lower than today's doses are very cost-effective in reducing overall drug manufacturing costs. It is contemplated that the direct delivery of nintedanib to the deep lung of the patient improves the overall action as a non-specific tyrosine kinase inhibitor as compared to oral administration. As so contemplated, this will significantly reduce the amount of drug required to achieve an efficacious result using direct delivery as compared to oral delivery, resulting in improved targeting and tolerability.

In some embodiments, this disclosure provides for a dry powder composition suitable for direct pulmonary administration, which comprises particles comprising:
a) an effective amount of a pharmaceutical selected from pirfenidone and/or nintedanib;
b) a di-fatty acid phosphatidyl choline compound in an amount sufficient to impart stability to said particles; and
c) an effective amount of the divalent cation (e.g., calcium chloride),
   wherein said powder composition comprises particles having an average particle size (diameter along the longest axis) of from about 0.5 to about 15 μm; and a bulk density of from about 0.03 g/cc to about 0.30 g/cc.

In some embodiments, this disclosure provides for a dry powder composition suitable for direct pulmonary administration, which comprises particles comprising:
d) an effective amount of a pharmaceutical selected from pirfenidone and/or nintedanib;
e) a di-fatty acid phosphatidyl choline compound in an amount sufficient to impart stability to said particles; and
f) an effective amount of calcium chloride,
   wherein said powder composition comprises particles having an average particle size (diameter along the longest axis) of from about 2 to about 6 μm; and a bulk density of from about 0.04 g/cc to about 0.11 g/cc.

In some embodiments, any water present in said composition comprises a hydrate or a semi-hydrate of one or more of the starting materials used. Optionally, water can also be associated with the lipid or other excipients, and not present in a stoichiometric hydrate.

In some embodiments, the dry powder compositions described herein comprise combinations of two or more actives, said actives are uniformly dispersed throughout in a ratio proportional to the amount of each used in the composition on a weight basis.

In some embodiments, the relative weight ratio of pirfenidone to nintedanib in the dry powder compositions disclosed herein is from about 8:1 to about 40:1, from about 8:1 to about 100:1, from about 8:1 to about 80:1, about 10:1 to about 70:1, about 12:1 to about 60:1, about 15:1 to about 50:1, about 15:1 to about 40:1, about 20:1 about 40:1, and about 20:1 to about 30:1. In some embodiments, the relative weight ratio of pirfenidone to nintedanib in the dry powder compositions disclosed herein is from about 8.5:1 to about 40:1; in other embodiments, the ratio of pirfenidone to nintedanib in the dry powder compositions disclosed herein is from about 9:1 to about 30:1 and preferably 25:1. These ratios increase the amount of pirfenidone relative to nintedanib. It is contemplated that direct delivery of these drugs to the deep pulmonary epithelium at or proximate to the alveoli will reduce the amount of each drug required to achieve a therapeutic result by targeted delivery to the healthy alveolar cells and close, if not direct targeting of the fibrotic cells. In either case, absorption through the gastrointestinal tract is avoided to a great extent but not entirely, as are the multiple systemic side effects associated with the very high body exposures from the oral products.

In a preferred embodiment, the dry powder compositions described herein comprise a weight ratio of pirfenidone to nintedanib of about 10:1, about 15:1, about 20:1, about 25:1, about 30:1, about 35:1, or about 40:1, and any value between 10 and 40. Optionally, a dry powder composition as described herein comprises a weight ratio of pirfenidone to nintedanib of about 25:1.

In some embodiments, the dry powder compositions described herein can comprise one or more pharmaceutically acceptable additives. When used in this manner, these additives include one or more dispersing agents, a buffering agent, a disaccharide, an antioxidant, a stabilizer, a taste-masking agent, and any combination thereof.

In some embodiments, a dispersing agent is selected from leucine or trileucine, optionally in combination with each other, and each or both optionally in the presence of a further dispersing agent. In some embodiments, the further dispersing agent is selected from Tween 80® (polyethylene glycol sorbitan monooleate).

In some embodiments, the biocompatible buffer is selected from acetate, citrate, phosphate, tartrate, and triethanolamine buffers.

In some embodiments, the disaccharide is trehalose, lactose (anhydrate or monohydrate), or mannitol.

In some embodiments, the antioxidant is selected from ascorbic acid, butylated hydroxyanisole, butylated hydroxytoluene, cysteine, glutathione, propyl gallate, sodium metabisulfite, sodium thiosulfate, methionine, and Vitamin E.

In some embodiments, this disclosure provides for a method for treating a patient with pulmonary fibrosis, which method comprises:
a) confirming that the patient is currently diagnosed with either incipient or moderately developed progressive pulmonary fibrosis or idiopathic pulmonary fibrosis;
b) administering directly to the lungs of said patient a composition comprising a dry powder composition which itself comprises particles comprising:
i) an effective amount of a pharmaceutical combination of pirfenidone and, in some contemplated embodiments, optionally an effective amount of treprostinil;
ii) a di-fatty acid phosphatidyl choline compound in an amount sufficient to impart structural stability to said particles; and
iii) an effective amount of calcium chloride,
   wherein said particles of said powder composition have an average particle size (diameter along the longest axis) of from about 0.5 to about 15 μm or about 5.0 to about 15 μm; a bulk density of from about 0.03 g/cc to about 0.15 g/cc or about 0.04 g/cc to about 0.11 g/cc.

In some embodiments, patients being treated with the methods described herein exhibit a reduction in the rate of disease progression as compared to patients not undergoing said pharmacotherapy. In some embodiments, said reduction in the rate of progression is at least 10%, or at least 20%, or at least 30%, or at least 50%, or at least 75% less than that rate of progression of said disease for patients not treated with the methods described herein. The rate of progression may refer to measurements of Forced Vital Capacity (FVC) and/or 6-minute walk distance, as well as other indicators used in Phase 2b clinical studies.

In some embodiments, this disclosure provides for a method for qualifying a patient for pharmacotherapy treatment of idiopathic pulmonary fibrosis with a composition as described herein, which method comprises:
a) selecting a patient diagnosed with pulmonary fibrosis;
b) evaluating said patient to determine the stage of the disease for that patient;
c) disqualifying patients from said pharmacotherapy who are currently diagnosed as being in either Stage 3 or Stage 4 for oxygen requirements of the disease;
d) initiating pharmacotherapy for patients who are determined to be at the incipient stage of the disease (Stage 1 of oxygen requirements) or at a moderate stage of the disease (Stage 2 of oxygen requirements);
e) monitoring said patients to determine the extent of progression of said disease after initiating pharmacotherapy; and
f) optionally removing said patients from pharmacotherapy for whom the attending clinician determines that pharmacotherapy is no longer effective.

In some embodiments, this disclosure provides for a method for treating qualified patients diagnosed with pulmonary fibrosis, which method comprises:
a) confirming that said patient has been qualified for said pharmacotherapy with a composition as described herein;
b) initiating pharmacotherapy for said patient;
c) maintaining said pharmacotherapy until the attending clinician determines that the disease progression has evolved such that there is no longer a benefit to the patient; and
d) terminating said pharmacotherapy.

In some embodiments, the attending clinician can ascertain the degree of progression of the disease based on the PF scale, which is discussed herein. In some embodiments, the attending clinician can ascertain the degree of progression of the disease based on the increased rate of usage of oxygen.

In some embodiments, devices suitable for pulmonary delivery of dry powder compositions can include metered-dose inhalers (MDI) and dry powder inhalers (DPI) (i) manufactured by Chiesi, Merxin LTD, Plastiape S.p.A. (now part of Berry Global), and Emphasys Innovatec; (ii) described herein; and (iii) described in, for example, U.S. Pub No. 20250108180; U.S. Pat. Nos. 12,171,934, 10,994,083, and 11,185,647 each of which is incorporated herein by reference in its entirety.

In some embodiments, this disclosure further provides a method for treating a patient with pulmonary fibrosis, pulmonary arterial hypertension (PAH), or pulmonary hypertension in interstitial lung disease (PH-ILD, also known as progressive fibrosing interstitial lung disease), and which method comprises:
a) confirming that the patient is currently diagnosed with pulmonary fibrosis, pulmonary arterial hypertension (PAH), or pulmonary hypertension in interstitial lung disease (PH-ILD);
b) administering directly to the lungs of said patient a composition comprising a dry powder composition which itself comprises particles and:
   i) an effective amount of a pharmaceutical combination of pirfenidone and at least one additional therapeutic agent that can be administered by oral inhalation;
   ii) a di-fatty acid phosphatidyl choline compound in an amount sufficient to impart structural stability to said particles; and
   iii) an effective amount of calcium chloride; and
      wherein said powder composition comprises particles having an average particle size (diameter along the longest axis) of from about 0.5 to about 15 μm or about 5.0 to about 15 μm; a bulk density of from about 0.03 g/cc to about 0.30 g/cc or about 0.04 g/cc to about 0.11 g/cc. In some embodiments, the additional therapeutic agent is nintedanib. In some contemplated embodiments, the additional therapeutic agent is selected from nintedanib, treprostinil palmitil, seralutinib, and combinations thereof.

In some embodiments, provided is a composition comprising a dry powder composition which itself comprises particles comprising a) an effective amount of a pharmaceutical combination of i) pirfenidone and/or nintedanib and ii) treprostinil palmitil, that can be administered by oral inhalation; b) a di-fatty acid phosphatidyl choline compound in an amount sufficient to impart structural stability to said particles (i.e., stabilized); and c) an effective amount of a divalent cation (e.g., magnesium) in the form of a pharmaceutically acceptable salt. In an embodiment, the dry powder composition comprises particles having an average particle size (diameter along the longest axis) of from about 0.5 to about 15 μm or about 2 to about 6 μm; a bulk density of from about 0.03 g/cc to about 0.30 g/cc or about 0.04 g/cc to about 0.11 g/cc. Also provided is a method for treating a patient with pulmonary fibrosis, pulmonary arterial hypertension (PAH), or pulmonary hypertension in interstitial lung disease (PH-ILD), the method comprising: a) confirming that the patient is currently diagnosed with pulmonary fibrosis, pulmonary arterial hypertension (PAH), or pulmonary hypertension in interstitial lung disease (PH-ILD); and b) administering directly to the lungs of the patient the dry powder composition as describe above.

In some embodiments, provided is a dry powder composition comprising nintedanib with the proviso that the dry powder composition does not contain any lactose (optionally lactose monohydrate) or fumaryl diketopiperazine (FDKP) as a carrier. Also provided is a dry powder composition comprising an effective amount of first medicament which is a longer chain ester form of nintedanib having —C₈₋₂₀ alkyl as R group, optionally having —C₁₆ alkyl as R group, optionally a second medicament selected from treprostinil, treprostinil palmitil, seralutinib, or pirfenidone, or a pharmaceutically acceptable salt thereof, and which further comprises a lecithin component, optionally a di-fatty acid phosphatidyl choline.

In some embodiments, provided is a method of preparing a dry powder composition comprising pirfenidone and nintedanib or a pharmaceutically acceptable salt thereof, the method comprising: a) emulsifying a suspension medium comprising pirfenidone and nintedanib or a pharmaceutically acceptable salt thereof, optionally the di-fatty acid phosphatidyl choline and calcium chloride or magnesium chloride, in a volatile solvent to form a stable nanoemulsion; b) atomizing the stable nanoemulsion into a drying chamber, wherein heated drying gas removes the volatile solvent, thereby obtaining the dry powder composition, wherein an outlet temperature is controlled at no more than 72° C., no more than 71° C., no more than 70° C., no more than 69° C., no more than 68° C., no more than 67° C., or no more than 66° C., or no more than 65° C.

### BRIEF DESCRIPTION OF THE FIGURES

FIG. 1 depicts an exemplary process of making a suspension containing the APIs.

FIG. 2 depicts the recovery of pirfenidone as a function of spray-dryer outlet temperature as described in Example 17.

FIG. 3 depicts a TGA thermogram of pirfenidone, wherein the pirfenidone sample was heated using a ramp of 5° C./min to 300° C. as described in Example 17.

FIG. 4 depicts a multi-step TGA experiment in which pirfenidone was heated in 10° C. increments from 50° C. to 140° C., holding isothermally for 15 min at each 10° C. temperature increment, as seen in the stepwise shifts, as described in Example 17.

FIG. 5 depicts the Arrhenius plot of isothermal mass loss rates of pirfenidone (measured using a stepwise isothermal TGA experiment) as described in Example 17. The vertical dotted line corresponds to the melting temperature of pirfenidone.

FIG. 6 depicts a TGA thermogram of nintedanib esylate (NE) as described in Example 18.

FIG. 7 depicts first and second DSC heating scans of Nintedanib esylate as described in Example 18.

FIG. 8 depicts an overlay of DSC and TGA heating scans of Nintedanib esylate as described in Example 18.

FIG. 9 depicts the first DSC heating scan of Nintedanib esylate as described in Example 18.

FIG. 10 depicts the second DSC heating scan of Nintedanib esylate as described in Example 18.

FIG. 11 depicts the first-heating DSC scan of pirfenidone as described in Example 18.

FIG. 12 depicts the second-heating DSC scan of pirfenidone (enlargement of Tg and recrystallization region) as described in Example 18.

FIG. 13 depicts the second-heating DSC scan of pirfenidone as described in Example 18.

FIG. 14 depicts XRPD patterns of nintedanib esylate, nintedanib free base, and pirfenidone as described in Example 19.

FIG. 15 depicts the low-angle region of the X-ray powder patterns of nintedanib esylate, nintedanib free base, and pirfenidone as described in Example 20.

FIG. 16 depicts XRPD patterns of nintedanib free base, recrystallized using nintedanib esylate as the starting material as described in Example 21. The powder pattern of the nintedanib esylate starting material is shown for reference.

FIG. 17 depicts the first DSC heating scan of nintedanib free base as described in Example 21.

FIG. 18 depicts the second DSC heating scan of nintedanib free base following melt quenching in the DSC as described in Example 21.

FIG. 19 depicts the TGA thermograms of two different preparations of nintedanib free base as described in Example 21.

### DETAILED DESCRIPTION

Disclosed are compositions and methods for treating incipient, mild, or moderate pulmonary fibrosis and idiopathic pulmonary fibrosis (PF) (collectively referred to herein as pulmonary fibrosis or “PF”) in patients in need thereof. Prior to describing the compositions and methods in more detail, the following terms will be defined.

### Acronyms and Definitions

6 MWT 6 min walk test
μg microgram when associated with a number
μL or μl microliter when associated with a number
μm micrometer when associated with a number
AE adverse event
API active pharmaceutical ingredient
Cc cubic centimeter
CSF1R colony-stimulating factor 1 receptor
DLCO lung diffusion capacity for carbon monoxide
DPI dry powder inhaler
DSPC distearoylphosphatidylcholine
FEV1 Forced Expiratory Volume in 1 second
FVC Forced Vital Capacity
g gram when associated with a number
GERD gastroesophageal reflux disease
IPF idiopathic pulmonary fibrosis
MDI metered dose inhaler
mg milligram when associated with a number
min, Min, or min. minute when associated with a number
mL or ml milliliter when associated with a number
mol mole
M molar
PAH pulmonary arterial hypertension
PC phosphatidylcholine
PDGFR-alpha (PDGFR-α) platelet-derived growth factor receptor-alpha
PDGFR-beta (PDGFR-β) platelet-derived growth factor receptor-beta
PF pulmonary fibrosis
PFOB perfluorooctyl bromide
PH-ILD pulmonary hypertension in interstitial lung disease also referred to as progressive fibrosing interstitial lung disease
pMDI pressurized metered-dose inhalers
ppb parts per billion
PPF progressive pulmonary fibrosis
rpm rotations per minute
S second when associated with a number
SCFR stem cell growth factor receptor kinases
SM suspension medium
SSC-ILD systemic sclerosis-associated interstitial lung disease
Tm melt transition temperature
TOC total organic carbon
wt % weight percent

The following definitions provide clarity to certain terms and phrases used in this application. It is understood that terms or phrases used herein that are not defined have their accepted medical or biological meaning.

The term a “dry powder” refers to a powder that possesses a moisture content that allows the powder to remain chemically and physically stable during storage at ambient temperature and which is easily dispersible. In some embodiments, the dry powders disclosed herein will have no more than about 6 weight percent water in the dry powder as determined by the weight differential of the dry powder before and after desiccation. In some embodiments, the amount of water in said dry powder is from about 0.2 to about 6.0 weight percent water, as determined in this manner.

“Suspension medium” refers to certain fluids that, when combined with water and a distearoylphosphatidylcholine or other di-fatty phosphatidylcholine, form a stable emulsion to which the active(s) and calcium dichloride can be added. In this regard, such suspension media include those set forth in U.S. Pat. No. 9,554,993, which is incorporated herein by reference in its entirety. As above, the suspension medium is maintained at a temperature of no more than 60° C.

The terms “admix”, “admixed”, and “admixing” refers to the act of mixing, generally two or more components forming a new composition.

The terms “administering”, “administration”, and “administer” mean to dispense, provide, and/or apply, and refer to any route of administration of a compound. For example, administering can refer to respiratory inhalation administration to a subject, such as oral inhalation and nasal inhalation. In some embodiments, administration is by respiratory inhalation. In some embodiments, respiratory inhalation is oral inhalation.

Administration can be contemporaneous or sequential. By the term “sequential” is meant that a drug is administered one after another in any order. Sequential administration can be within a few minutes of each other but separated by more than 5 minutes, or separated by a few hours. By “contemporaneous” is meant occurring at the same time. For example, two or more drugs are administered contemporaneously when in they are in the same formulation or within 5 minutes of each other.

The terms “drug”, “active pharmaceutical ingredient”, “medicament”, “medication”, and “drug active” as used interchangeably herein and refer to an individual active compound used in a combination therapy or combination drug therapy as described herein.

When referring to ratios and ranges of a drug in a relative weight ratio, the ratios are expressed as the free base of the drug. The range weight ratio of a drug's esylate, chloride, or other form, for example, differs from the free form.

As used herein, a “combination therapy”, and “combination drug therapy” are used interchangeably to refer to a therapeutic method involving two or more different treatments, often two or more different medications, to treat a single condition. In a combination therapy, two or more medications can be administered sequentially or simultaneously in one unit or more than one unit. A combination therapy, for example, can include a triple drug therapy.

The term “reduction” or “reducing” refers to the extent to which a composition, as described herein, impacts the progression of PF in patients so treated as compared to patients not so treated, where both sets of patients started at an equivalent point in time. In some embodiments, the reduction in the rate of disease progression can be at least about 10%, or at least about 20%, or at least about 30%, or at least about 40%, or at least about 50%, or at least about 75%, for treated patients to reach a certain milestone in disease progression as compared to equivalent patients who are untreated with the compositions described herein. It is understood that this rate of disease progression is an average of treated patients, compared with an average of untreated patients. In some instances, it is contemplated that, for two cohorts of equal numbers of treated and untreated patients at an equivalent stage of moderate disease progression, untreated patients at Stage 2 may progress to Stage 3 in 24 months. In contrast, the treated patients would arrive at that stage in 26.4 months (10% reduction), or in 28.8 months (20% reduction), or in 36 months (50% reduction) to reach that milestone point in disease progression.

“Patient” and “subject” are used herein interchangeably and refer to any animal (e.g., a mammal, such as a human, a laboratory animal, such as a mouse, rat, rabbit, guinea pig, or other animal models of idiopathic pulmonary fibrosis, or a domesticated animal, such as a dog, cat, or a domesticated animal, for example, sheep, horses, cattle, pigs and goats). A subject in need of treatment, according to the methods described herein, may be one who has been diagnosed with or otherwise qualified as having incipient, mild, or moderate PF. Preferably a patient is qualified as having incipient, mild or moderate PF. The patient may be diagnosed as having systemic sclerosis-associated interstitial lung disease (SSc-ILD) or pulmonary hypertension in interstitial lung disease. SSc-ILD is a progressive autoimmune lung condition that is a complication of systemic sclerosis. SSc-ILD causes the patient to have inflammation and scarring (fibrosis or fibrosing) lung tissue, leading to shortness of breath, dry cough, fatigue, and reduced oxygen exchange.

By “pharmaceutically acceptable salts” is meant to include salts of the compounds listed herein, which are prepared with relatively non-toxic acids or bases, depending on the particular substituents found on the compounds described herein. When compounds described herein contain relatively acidic functionalities, base addition salts can be obtained by contacting the neutral form of such compounds with a sufficient amount of the desired base, either neat or in a suitable inert solvent. Non-limiting examples of salts derived from pharmaceutically acceptable inorganic bases include aluminum, ammonium, calcium, copper, ferric, ferrous, lithium, magnesium, manganic, manganous, potassium, sodium, zinc, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively non-toxic organic acids like acetic, propionic, isobutyric, malonic, benzoic, succinic, suberic, fumaric, mandelic, phthalic, benzenesulfonic, p-tolylsulfonic, citric, tartaric, ethanesulfonic, methanesulfonic, and the like. Also included are salts of amino acids, such as arginate and the like, and salts of organic acids like glucuronic or galacturonic acids and the like. See, e.g., Berge, S. M., et al., “Pharmaceutical Salts” J. Pharm. Sci., 1977, 66(1): 1-19; see also P. H. Stahl and C. G. Wermuth, editors, HANDBOOK OF PHARMACEUTICAL SALTS: PROPERTIES, SELECTION AND USE, Weinheim/Zurich: Wiley-VCH/VHCA, 2002; the disclosure of each is incorporated herein by reference in its entirety.

As used herein, a “pharmaceutically acceptable cocrystal” refers to a crystalline material formed by combining a compound (e.g., pirfenidone) and one or more relatively non-toxic cocrystal formers (i.e., a molecule). In certain instances, cocrystals may have improved properties as compared to the parent form (i.e., the free molecule). Improved properties can be increased solubility, increased dissolution rate, increased bioavailability, increased dose response, decreased hygroscopicity, decreased form diversity, more desired morphology, and the like. Methods for making and characterizing cocrystals are known to those of skill in the art.

As used herein, a “taste-masking agent” is a pharmaceutically acceptable agent that can mask the bitter taste of an active component (e.g., pirfenidone) in a pharmaceutical composition, other unpleasant tastes present in the pharmaceutical composition, or any combination thereof. One or more taste-masking agents can be present in the oral pharmaceutical composition. Exemplary taste-masking agents may include, but are not limited to, sweeteners, flavoring agents, synthetic flavors, bitter blockers, complexing agents, and amino acids or salts thereof. See, e.g., U.S. Pat. No. 5,633,006. Sweeteners can be added to formulations to counteract bitterness and enhance palatability. Sweeteners may be artificial sweeteners such as aspartame, sucralose, saccharinic acid, and acesulfamic acid or salts thereof; natural sweeteners such as sucrose, fructose, and glucose; or sugar alcohols (polyols) such as mannitol, sorbitol, xylitol, erythritol, and maltitol. Flavoring agents may be natural flavors such as peppermint oil, spearmint oil, citric acid, vanilla, raspberry juice, and licorice extract, or synthetic flavors such as ethyl maltol, ethyl vanillin, and maltol. Bitter blockers may be sodium acetate, sodium gluconate, adenosine 5′-monophosphate (AMP), and homoeriodictyol (from Yerba Santa). Complexing agents may be cyclodextrins such as beta-cyclodextrin, gamma-cyclodextrin, and hydroxypropyl beta-cyclodextrin. Amino acids suitable for being used as taste-masking agents may be glycine, alanine, taurine, and glutamic acid.

“Therapeutically effective amount”, “therapeutic amount,” and “pharmaceutically effective amount” are used interchangeably and refer to a non-toxic but sufficient amount of a composition as described herein to provide the desired biological result(s), and/or to an amount sufficient to carry out a specifically stated purpose.

“Treatment”, “treating”, “treatment of”, and other grammatical uses of the term “treat” are used interchangeably and refer to an approach for obtaining beneficial or desired results, including clinical results. Beneficial or desired clinical results can include, but are not limited to, at least a partial alleviation or partial amelioration of one or more symptoms or conditions; at least a partial diminishment of the extent of the condition, disorder or disease; at least a partial stabilization of the state of condition, disorder, or disease; prevention or reduced likelihood of developing a condition, disorder, or disease; prevention or reduced likelihood of spreading a condition, disorder, or disease; at least a partial delay or slowing of a condition, disorder, or disease progression; at least partially delay or slowing the progression of a condition, disorder, or disease, or the onset thereof. Methods for determining the result of a treatment are known in the art.

For claim construction in the United States only, the terms “prefer,” “preferred,” “preferably”, and “prefer” should be omitted from the sentence when interpreting the claims.

As used herein, when a range is described, that range includes both the endpoints of the range as well as all numbers in between. For example, “between 1 mg and 10 mg” includes 1 mg, 10 mg, and all 0.1 mg amounts between 1 mg and 10 mg. In some embodiments, the amounts included in such ranges are rounded to two decimal points when the recited value is defined in 3 or more decimal points or to one decimal point when the recited value is defined in 2 or more decimal points. For example, rounding to two decimal points would entail that any value in the third decimal point that is less than “5” is lowered to the two decimal point value recited, and any value in the third decimal point that is “5” or higher raises the value of the second decimal point to the next higher number.

As used herein, the singular forms “a”, “an”, and “the” are intended to include the plural forms as well, unless the context clearly indicates otherwise.

As used herein, the term “about” when used before a numerical designation, e.g., temperature, time, amount, concentration, and such other, including a range, indicates approximations that may vary by (+) or (−) 10%, 5%, 1%, or any subrange or a sub value therebetween. In some embodiments, the term “about” when used with regard to a dose amount means that the dose may vary by +/−10%.

As used herein, “direct pulmonary delivery” is synonymous with “oral inhalation.”

As used herein, the term “deep pulmonary tract” refers to the tissue adjacent to or proximate to the alveoli in the lungs, which, correspondingly, will be proximate to the fibrotic tissue. The lower respiratory zone is located within the chest, specifically encompassing the small bronchioles, alveolar ducts, and 300-500 million alveoli where gas exchange occurs. The deep pulmonary tract is sometimes referred to as the respiratory zone to distinguish it from the conducting zone (nose to bronchi) that merely moves air in and out.

As used herein, the term “comprising” or “comprises” is intended to mean that the compositions and methods include the recited elements, but do not exclude other non-recited elements.

A composition or method with the transitional phrase “consisting essentially of” the essential elements, compounds, compositions, or steps as described herein, but does not not exclude other materials or steps that do not materially affect the basic and novel characteristic(s) of the claimed disclosure.

As used herein, the term “consisting of” shall mean excluding more than trace elements of other ingredients and substantial method steps. Embodiments defined by each of these transition terms are within the scope of this disclosure.

As used herein, the term “propellant” refers to conventional agents used to deliver a dry powder composition from a dispenser, such as an MDI inhaler, into the deep lung tissue, with a portion being delivered to the alveoli and adjacent tissue.

As used herein, the term “pirfenidone” refers to a compound represented by formula I:

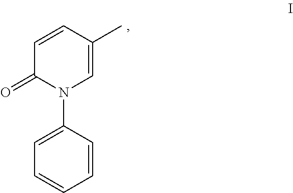

as well as its tautomeric structure I-A:

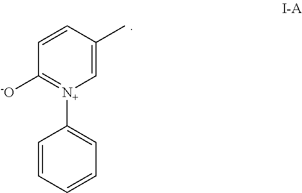

The term pirfenidone also encompasses deuterated forms of pirfenidone such as

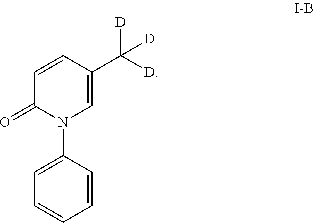

as well as its tautomeric structure as shown above. This compound (I-B) is sometimes referred to as deupirfenidone, LYT-100, developed by PureTech. A pharmaceutically acceptable cocrystal of pirfenidone may also be used. Exemplary cocrystals of pirfenidone are described in Meng et al., CrystEngComm, 2023, 25:5133-5140. For the dry powder compositions described herein, a crystalline form of pirfenidone may be advantageous. In the dry powder compositions described herein, more than about 70%, 80%, or 90% of pirfenidone may be present as a crystalline form. It can also be possible that the dry powder may have lower crystallinity or even amorphous pirfenidone present.

As used herein, the term “nintedanib” refers to a compound represented by formula II, wherein R is —C₁₋₂₀ alkyl, preferably methyl, ethyl, or —C₁₆ alkyl, in a free base form. The term “nintedanib” also includes pharmaceutically acceptable salts thereof. One representative, non-limiting example of such a salt is “nintedanib esylate”, which refers to the esylate salt as per formula II-A, where R is —C₁₋₂₀ alkyl, preferably methyl, ethyl, or —C₁₆ alkyl:

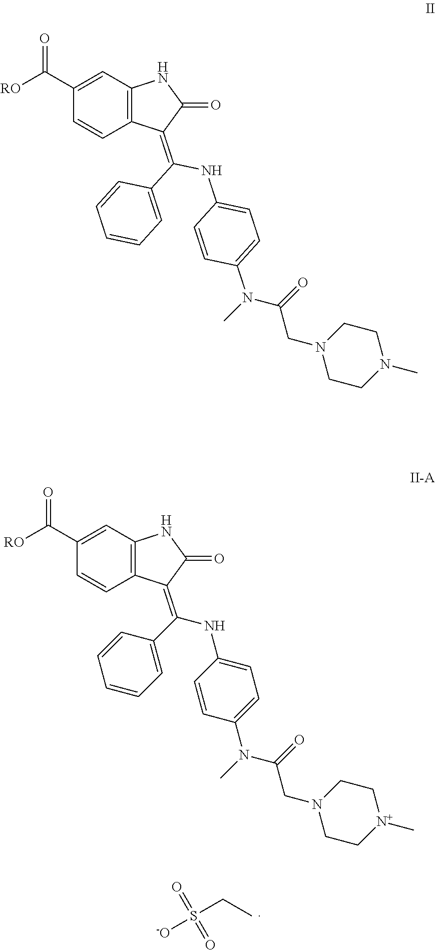

It is contemplated that the longer chain ester forms (e.g., R is —C₈₋₂₀ alkyl; preferably R is —C₁₆ alkyl [palmitil]) could provide for sustained activity, thereby providing for extended interval dosing of nintedanib treatments.

As used herein, the term “treprostinil” refers to a compound represented by formula III:

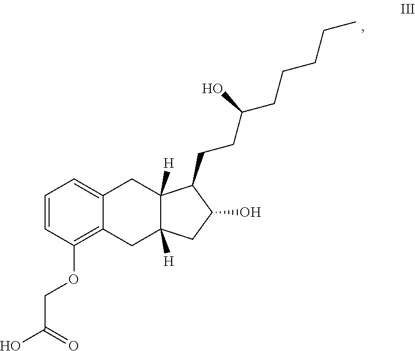 [SMILES: CCCCC[C@H](O)CC[C@@H]1[C@H]2Cc3cccc(OCC(=O)O)c3C[C@H]2C[C@H]1O]

which includes pharmaceutically acceptable salts thereof. “Treprostinil sodium” refers to a compound represented by formula III-A:

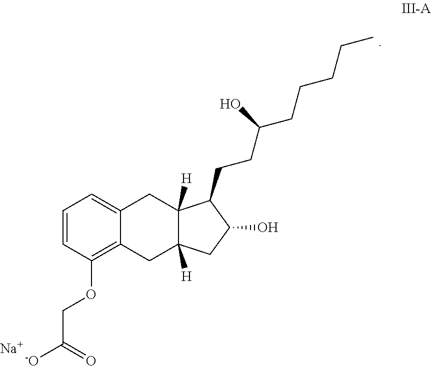

As used herein, the term “treprostinil palmitil” refers to a compound represented by formula IV:

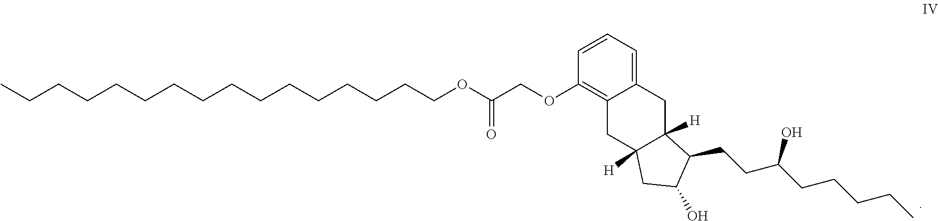 [SMILES: CCCCCCCCCCCCCCCCOC(=O)COc1cccc2c1C[C@H]1C[C@@H](O)[C@H](CC[C@@H](O)CCCCC)[C@H]1C2]

As used herein, the term “seralutinib” refers to a compound represented by formula V:

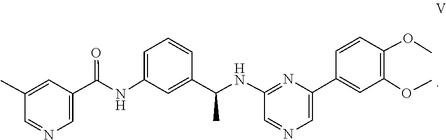

As used herein, the term “incipient PF” refers to patients who are newly diagnosed with a pulmonary fibrosis condition and whose disease is characterized by the attending clinician as Stage 1. Specifically, Stage 1 refers to patients who meet the following criteria for purposes of treatment using the compositions and methods described herein.

### Stage 1: Recently Diagnosed

A patient is first diagnosed with PF who has been tested for Pulmonary Function or Lung Function Tests to gauge how much air the lungs of the patient can hold and who may have early symptoms of PF, such as one or more fatigue, shortness of breath, and a distinctive, dry cough. Incipient PF patients do not require oxygen at this stage of PF, either while resting or during limited activity.

As used herein, the term “moderate PF” refers to patients diagnosed with PF and whose disease is characterized as Stage 2. Specifically, Stage 2 PF refers to patients who meet the following criteria who meet the following criteria for purposes of treatment using the compositions and methods described herein:

### Stage 2: Oxygen is Needed Only with Activity

Patients clinically identified with moderate PF are those who are determined by a clinician to be at Stage 2 of PF. At this stage, a PF patient experiences coughing more frequently, shortness of breath with activity, and is prescribed oxygen. Some patients at this stage can use a pulse oximeter to monitor their body's oxygen levels, i.e., equal to or greater than 89%. Such oxygen administration is provided to avoid hypoxemia (the inability to get enough oxygen into the blood, resulting in low oxygen levels).

As used herein, the term “severe PF” refers to patients diagnosed with PF whose disease is characterized clinically as Stage 3 or Stage 4 based on their oxygen requirements. Patients determined to have Stage 3 PF meet the following criteria for purposes of treatment using the compositions and methods described herein:

### Stage 3: Needing Oxygen Throughout the Day

A patient is characterized as having Stage 3 PF if the patient experiences shortness of breath with and without activity, and low oxygen levels at rest (i.e., below 89% without oxygen). Cough and fatigue at this stage are bothersome. Patients at this stage require oxygen throughout the day to avoid hypoxemia and even hypoxic respiratory failure.

### Stage 4: Advanced Oxygen Needs

As PF progresses, Stage 4 is reached when a portable oxygen delivery system is no longer sufficient to meet the patient's needs, and the patient is placed on a high-flow oxygen delivery device. At this stage, the disease is approaching the terminal stage, and very little in the way of therapy is available to ameliorate the patient's condition.

A patient's PF classification may also or alternatively be based on FEV1 (Forced Expiratory Volume in 1 s), FVC (forced vital capacity), and an indirect measure referred to as the 6 minute walk test (6 MWT).

In all cases, the ultimate decision as to the classification of the patient's PF is provided by the attending clinician.

As used herein, the term “uniform” or “uniformly” as used herein to describe the concentration of each anti-fibrotic agent in the dry powder composition is substantially the same (within +/−10%; preferably within +/−5%; and more preferably within +/−1%) of the average composition found for the entirety of the composition when different portions of the composition are tested for the concentration of actives therein.

As used herein, the term di-fatty acid phosphatidyl choline means that two of the three hydroxy groups on the glycerol backbone are substituted with two fatty acid esters, and a terminal hydroxy group is a choline phosphate ester. An example of a di-fatty acid phosphatidyl choline is distearoylphosphatidyl choline, which is depicted below as formula VI:

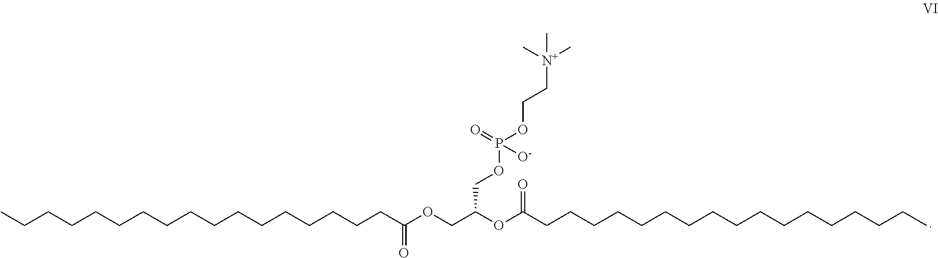 [SMILES: CCCCCCCCCCCCCCCCCC(=O)OC[C@H](COP(=O)([O-])OCC[N+](C)(C)C)OC(=O)CCCCCCCCCCCCCCCCC]

It is understood that other fatty acids can be used in place of stearic acid, which is representative only. For example, the fatty acid employed in said di-fatty acid phosphatidyl choline comprises a carboxyl group which forms an ester with the alpha and beta hydroxyl groups on the glycerol backbone, whereas the phosphatidyl choline forms on the delta hydroxyl group. The fatty acids can be C₉ to C₂₇ alkyl chains.

In some embodiments, the fatty acid employed is a long-chain saturated fatty acid. In some embodiments, the saturated fatty acid is a naturally occurring fatty acid containing from 8 to 28 carbon atoms, preferably from 15 to 22 carbon atoms. Table 1 below provides a list of some of the naturally occurring fatty acids.

TABLE 1

| Acid | Structure |
|---|---|
| Pentadecanoic | CH₃CH₂CH₂CH₂CH₂CH₂CH₂CH₂CH₂CH₂CH₂CH₂CH₂CH₂COOH |
| Palmitic | CH₃CH₂CH₂CH₂CH₂CH₂CH₂CH₂CH₂CH₂CH₂CH₂CH₂CH₂CH₂COOH |
| Stearic | CH₃CH₂CH₂CH₂CH₂CH₂CH₂CH₂CH₂CH₂CH₂CH₂CH₂CH₂CH₂CH₂CH₂COOH |
| Arachidic | CH₃CH₂CH₂CH₂CH₂CH₂CH₂CH₂CH₂CH₂CH₂CH₂CH₂CH₂CH₂CH₂CH₂CH₂CH₂COOH |
| Docosanoic | CH₃ CH₂CH₂CH₂CH₂CH₂ CH₂CH₂CH₂CH₂CH₂CH₂CH₂CH₂CH₂CH₂CH₂CH₂CH₂CH₂CH₂COOH |

As used herein, the term “dry powder” refers to a fine particulate composition prepared by the methods described herein. The dry powder is not suspended or dissolved in a propellant or other liquid. In one embodiment, the dry powder composition can contain up to about 6 weight percent water while retaining the aspects of an otherwise anhydrous material. Without being limited to any theory, the presence of a modest amount of water in the composition imparts stability to the dry powder composition and renders it resistant to water (e.g., substantially non-hygroscopic) at room temperature, thereby allowing for a long-term shelf-life. In one aspect, a “stabilized dry powder composition” as described herein is one that has a long-term (at least 90 days) shelf-life relative to its nebulizer counterpart composition.

The composition of this disclosure represents a paradigm shift in the treatment of PF. Rather than relying upon orally delivered drugs with their attendant side effects, the present compositions are designed to deliver the composition directly to the deep lungs, with a major portion being deposited at the alveoli and adjacent tissue. As the pathology of PF entails forming an oxygen-impermeable fibrotic scar over the alveoli, the progression of this disease continues until a sufficient portion of the alveoli is compromised and the patient dies. To more effectively treat this disease, therapy at the site of the disease must be integral to the process.

In some embodiments, the compositions described herein comprise pirfenidone and/or nintedanib (or its pharmaceutically acceptable salt), and in other contemplated embodiments, either or both optionally including treprostinil, treprostinil palmitil, or seralutinib. Each of these components, either alone or in combination, is delivered directly at the site of disease progression, thereby reducing the amount of drug(s) required to achieve a therapeutically effective outcome for the individual patient. It is contemplated that delivery to the site of disease progression (i.e., lung alveoli and surrounding tissues) will benefit the patient in one or more of the following:
the amount of drug needed will be substantially lower compared to that required for oral delivery, with significantly lower drug compound manufacturing costs;
the resulting gastrointestinal tract side effects accompanying oral delivery of pirfenidone and nintedanib, or contemplated combinations with other therapeutic agents, will be obviated or reduced to such an extent that the patient will stay on the therapy (e.g., improved medication compliance), given the substantially lower dosages administered, thereby improving tolerability;
higher patient compliance rates as compared to oral delivery and nebulized inhalation delivery;
a portion of the combination drug therapy comprising two or more active drug compounds (e.g., three active drugs) typically will be retained in the pulmonary tissue for a number of hours, with a lower amount passing through the alveoli and entering the systemic circulation. The blood circulating portion of the two-drug combination (or three-drug combination) will recirculate through the lungs but will also eventually be metabolized and eliminated without passing through the gastrointestinal tract;
the portion remaining in the lungs is not immediately subject to degradation by passage through the liver;
the drug combination therapy described herein can produce an increase in drug therapeutic efficacy and perhaps an increase in drug half-life in the patient receiving the combination therapy;
the combination therapy can achieve a peak drug concentration in the lungs that is significantly higher at the site of disease progression than what can be achieved by oral delivery to the patient;
the combination therapy described herein for inhalation will have enhanced stability relative to a nebulizer formulation and nebulized delivery of the two drugs (or contemplated three or more drugs);
the combination therapy will decrease a patient's cough frequency over time;
the combination therapy will decrease a patient's coughing that generally occurs immediately after inhalation of the composition using traditional nebulizer formulations; and
the drug combination of the dry powder formulations described herein will be substantially non-agglomerating when the patient inhales the powder out of the device, which can greatly improve deep lung deposition following inhalation.

In a nintedanib-pirfenidone combination, a therapeutic endpoint sought is to prevent exacerbation of inflammation through pirfenidone, which reduces collagen growth factor production and release. Collagen development forms rope-like structures that form scar tissue, or fibrotic tissue, in the lungs. The addition of nintedanib, a non-specific tyrosine kinase inhibitor, may be synergistic with pirfenidone in reducing the overall inflammatory response and the development of additional fibrotic tissue, forming a second therapeutic endpoint sought with the dual therapy of inhaled drug therapy. An unexpected result is to achieve one or more of the benefits recited above and, herein, slowing PF progression or arresting PF progression, which to date cannot be obtained by existing drug therapies. The individual drug compound's chemical characteristics and pharmacokinetics in a patient compound the difficulty in formulating a combination that achieves the desired drug ratio, is stable, and can be predictably administered into a patient's lungs, let alone achieving any one or more clinical benefits. The indicated formulation and administration hurdles described exemplify the difficulty and unexpected path to achieve the combination and demonstrate its nonobviousness. Moreover, the combination meets a long-felt unmet need for patients with PF for better means and compositions to treat their condition.

In a contemplated embodiment, adding treprostinil palmitil or treprostinil to a nintedanib-pirfenidone combination may further increase the drugs' synergy, because treprostinil palmitil and treprostinil inhibit smooth muscle growth. In PF, pulmonary muscle growth creates a tissue foundation for the collagen deposits to take hold. Clinically, it is desirable if at least two times as much drug can be absorbed by a diseased lung cell via pulmonary administration of the drug than if delivered orally, even assuming the cellular half-life of the drugs administered orally and via inhalation are equivalent; currently such cellular uptake is not occurring in patients receiving oral administration of each agent in the form of a monotherapy.

Such clinical drug synergies are currently unavailable with the nintedanib-pirfenidone combination therapy (or the contemplated nintedanib-pirfenidone-treprostinil palmitil triple therapy) when administered orally, given the pharmacokinetics of the individual agents and their adverse effects in patients, leading to therapy discontinuation. Encapsulation of the drug combination (or contemplated triple drug/medicament therapy) into particles is made difficult, given the chemical differences in the drugs. Known adverse effects encountered when pirfenidone is used as a monotherapy include: diarrhea, nausea, vomiting, a skin ulcer, abdominal pain, an elevated liver enzyme level (e.g., aspartate transaminase, alanine transaminase, or gamma-glutamyl transpeptidase), patient weight decrease, fatigue, decreased patient appetite, a headache, pyrexia, back pain, dizziness, and hypertension.

Adverse events/effects (AEs) experienced by patients undergoing nintedanib monotherapy include: nausea, a rash, stomach pain, an upper respiratory tract infection, diarrhea, fatigue, a headache, indigestion, dizziness, decreased patient appetite or loss of appetite, gastroesophageal reflux disease (GERD), sinusitis, sleeplessness, weight loss, and joint pain.

In addition, it is contemplated that a combination of an effective amount of each of nintedanib and pirfenidone in the dry powder is more efficient and effective than serial delivery of a dry powder comprising only nintedanib, followed by or preceded by delivery of a dry powder comprising only pirfenidone. Without being limited to any theory, it is contemplated that the homogenous composition comprising both nintedanib and pirfenidone results in simultaneous and direct targeting of the interstitial region of the alveolar membrane. In contrast, separate administration introduces uncertainty of delivery to the intended site of action. Without being limited to any theory, serial administration may reduce the amount of the second drug delivered relative to the first, both in total and into the deep pulmonary tract.

### Methodology

As mentioned above, a dry powder composition is prepared from a combination comprising the active(s) with a di-fatty acid phosphatidyl choline, calcium chloride, water, and a dispersant or emulsifying agent such as a suspension medium. Such agents for use in dry powders are well known in the art. See, for example, Labiris, et al., Br. J. Clin. Pharmacol., 2003, 56(6): 600-612 and at column 40 of U.S. Pat. No. 9,554,993, which are incorporated herein by reference in their entirety. These agents are conventional.

The methods for preparing the dry powder compositions include those set forth by Labiris, et al., supra, as well as Chaurasiya, et al., “Dry Powder for Pulmonary Delivery: A Comprehensive Review,” Pharmaceutics, 13(1): 31 (2020), which is incorporated herein by reference in its entirety. The ability to make a dry powder for pharmaceutical purposes is recited at length therein and provides guidance as to the conditions that can be used to make dry powder compositions.

In one embodiment, the methods involve forming an emulsion comprising ultrapure water, a liquid suspension medium, and solids as described above and in the examples. In some embodiments, the process steps include combining PC and water and homogenizing the combination using a high-shear mixer. This can be followed by the addition of calcium chloride (CaCl₂)) while continuing to homogenize using a high-shear mixer. It is understood that magnesium chloride (MgCl₂) can be substituted for calcium chloride and is deemed to be an equivalent thereof.

Alternatively, calcium chloride (CaCl₂)) can be added to PC and water. In the instance wherein water is present, the water must be heated in order to disperse the lipid. This is a technique sometimes referred to as “melt dispersion” or “hot homogenization” as known in the art. The degree of heating depends on the lipid used. The suspension medium is then added while continuing to homogenize. The entire process is conducted at a temperature suitable for facilitating the formation of the stable nanoemulsion

In some embodiments, the relative amounts of ultrapure water and the liquid suspension medium range from about 8:1 to about 19:1 by weight. In some embodiments, the relative amounts of ultrapure water and the liquid suspension medium range from about 17:1 to about 19:1.

In some embodiments, the amount of anhydrous calcium chloride added is about 5.0 weight percent (wt %) or less based on the total weight of the solids used. In some embodiments, the amount of anhydrous calcium chloride added is about 4.0 weight percent or less based on the total weight of the solids used. In some embodiments, the amount of anhydrous calcium chloride added is from about 2.0 weight percent to about 7.0 weight percent based on the total weight of the solids used. The total weight of the solids used ranges from about 1.0 to about 3.0 weight percent of the weight of the cosolvent in the absence of such added solids. CaCl₂), as an emulsion stabilizer, can increase the main transition temperature of PC in the dry state. Typically, CaCl₂) can be added in a 1:2 mole ratio (mol Ca:mol PC).

In some embodiments, the amount of phosphatidyl choline added is about 2 to about 30 weight percent. In some embodiments, it can be about 30 to about 50 weight percent (wt %) based on the total weight of the solids used. In some embodiments, the amount of di-fatty acid phosphatidyl choline added is about 35 to about 45 weight percent based on the total weight of the solids used. In some embodiments, the amount of di-fatty acid phosphatidyl choline added is about 35 weight percent, or about 40 weight percent, or about 45 weight percent, or about 50 weight percent, or about 60 weight percent, or about 70 weight percent, or about 80 weight percent based on the total weight of the solids used.

The ultra-pure water is characterized as follows:

TABLE 2

| Resistivity | 18 megohms at 25° C. |
|---|---|
| Total organic carbon (TOC) | <5 ppb |
| Silica | <3 ppb |
| Sodium | <1 ppb |
| Chlorine | <1 ppb |

The water is further passed through a 0.20-micron filter to remove microbial contamination.

The drug(s) and emulsion are combined together and can be processed in a homogenizer to facilitate the formation of a stable emulsion-based feedstock. Once a stable feedstock is formed, it can be spray-dried to form a dry powder. The resulting dry powder can have a low or ultra low bulk density (approximately from about 0.03 g/cc to about 0.3 g/cc) and a uniform concentration of drug active or actives.

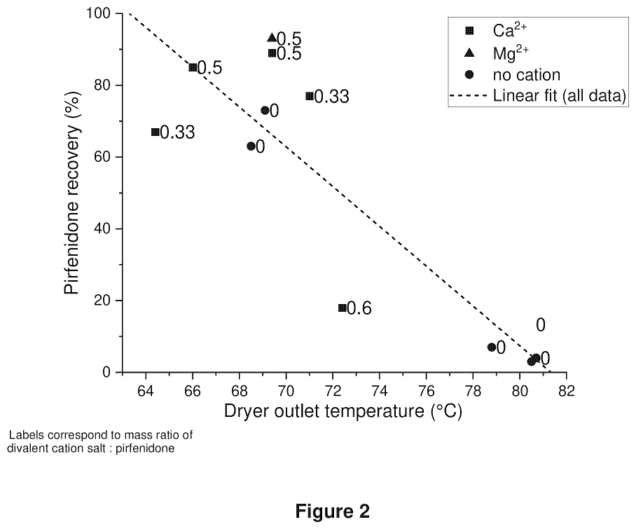

Spray drying is a particle engineering technique that can be used for the compositions described herein to manufacture respirable pharmaceutical powders. The technique has been demonstrated to work with both small molecules and biological actives, including proteins. For example, actives and excipients can be co-emulsified in a volatile solvent and then atomized into droplets that are sprayed into a drying chamber. Heated drying gas rapidly removes the solvent, resulting in a dried powder that is collected via cyclone or baghouse. The outlet temperature used during powder manufacture must be maintained such that the powder formed is not compromised. As shown in FIG. 2, when the outlet temperature is above about 72° C., the recovery of pirfenidone decreased significantly. As such, process conditions including the inlet temperature, the liquid fee rate, the drying gas flow rate, and other factors are necessarily controlled to provide for the required outlet temperature.

The spray-drying manufacture of an inhalation formulation with more than one active compound remains challenging. Two or more active pharmaceutical ingredients (APIs) can be combined into a single feedstock and spray dried. In addition to challenges associated with the chemical compatibility of the APIs, each individual API can have differing requirements for maximizing its physicochemical stability. Thus, coformulation of the APIs requires optimization. Alternatively, each active ingredient could be formulated and spray-dried separately and then blended together. This approach introduces additional processing steps and potential difficulties in content uniformity and is particularly challenging for inhalation powders, which have very poor flow and are often hygroscopic. See, e.g., K. B. Shephard et al., “Simultaneous Spray Drying for Combination Dry Powder Inhaler Formulations,” Pharmaceutics 14(6): 1130 (2022) and R. Vehring, “Pharmaceutical Particle Engineering via Spray Drying,” Pharm. Res. 25 (5): 999-1022 (2007).

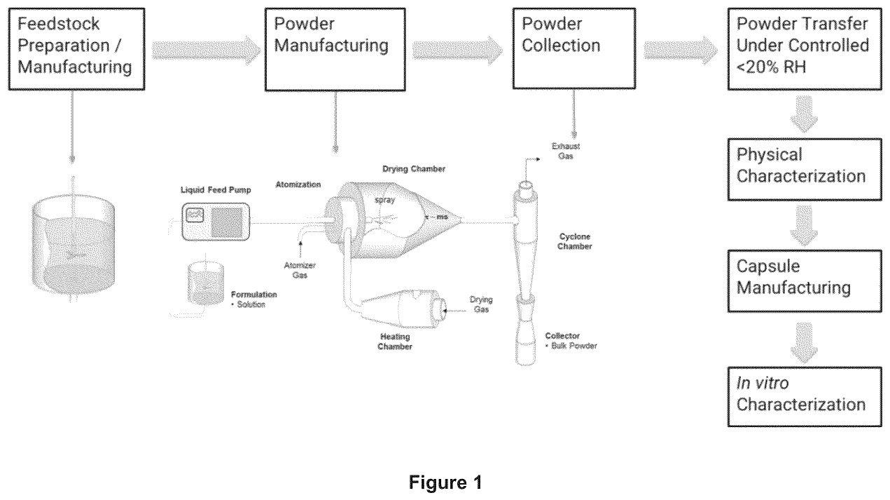

A process of making the suspension containing the APIs is depicted in FIG. 1.

In embodiments of the disclosure, the APIs (e.g., pirfenidone and nintedanib or a salt of nintedanib) are either dissolved or suspended in an aqueous solvent (e.g., a generally non-volatile solvent that is preferably pharmaceutically acceptable for use in the manufacture of pharmaceuticals even when the solvent is evaporated from the final product). In another embodiment, the drug is maintained in crystalline form and is not dissolved.

In some embodiments, a single dosage form or a portion of a single dosage is placed into a capsule for delivery by a conventional inhaler device.

### Inhalers

The dry powder compositions are delivered via a device such as a Turbuhaler®, an Accuhaler®, an Ellipta®, an Emphasys CDA-Haler, a Plastiape RS01 or RS02 dry powder inhaler. A Symbicort Turbuhaler® (AstraZeneca, UK) is a plastic device employing a breakable capsule that, once broken, is breath-activated. Following the directions provided, as the patient inhales, the dry powder is released and delivered into the patient's lungs.

The Accuhaler® (Glaxo Smith Kline, UK) is another example of a capsule-based inhaler similar to the Turbuhaler®. Other inhalers for use with the dry powder compositions are provided in, for example, U.S. Pat. Nos. 12,171,934, 10,994,083, and 11,185,647. In at least one embodiment, the dry powder compositions described herein can be delivered by a reservoir-based inhaler.

Generally, inhaling dry powder can induce coughing. When administered by oral inhalation, it is contemplated that the dry powder composition disclosed herein may induce less or no substantive coughing. This outcome would represent a marked improvement for therapeutic agents administered by oral inhalation.

### Methods

The compositions of this disclosure are useful in treating PF (e.g., IPF or PPF). Such methods comprise delivering via pulmonary inhalation an effective amount of the compositions suitable for inhalation as described herein (e.g., dry powder compositions). The methods can employ nintedanib or pirfenidone, especially if the attending clinician determines that a particular patient is ill-suited for treatment by both drugs. However, in some embodiments, a dry powder composition comprising both nintedanib and pirfenidone (and contemplated embodiments further comprising a treprostinil (e.g., treprostinil sodium, treprostinil palmitil, or seralutinib) is used to treat PF in a patient or other lung condition indicated herein. It is contemplated that when so used, the combination of nintedanib and pirfenidone as an example will benefit the patient because both drugs have a unique mechanism of action as compared to other drugs. Nevertheless, it is contemplated that such unique action by each drug may be synergistic.

In some embodiments, there is provided a method for treating a patient with PF, which method comprises:
a) confirming that the patient is currently diagnosed with either incipient, mild, or moderately developed idiopathic pulmonary fibrosis;
b) administering directly to the lungs of said patient a composition comprising a dry powder composition which itself comprises particles comprising:
i) an effective amount of a pharmaceutical combination of pirfenidone and optionally an effective amount of a treprostinil;
ii) an effective amount of a pharmaceutical combination of nintedanib and optionally an effective amount of a treprostinil.
iii) a di-fatty acid phosphatidyl choline compound in an amount sufficient to impart stability or aerosol performance, or powder dispersibility to said particles; and
iv) an effective amount of calcium chloride or magnesium chloride;

wherein said powder composition comprises particles having an average particle size (diameter along the longest axis or average geometric diameter) of from about 0.5 to about 15 μm, such as from about 5.0 to about 15 μm; and a low density of from about 0.03 g/cc to about 0.3 g/cc, such as about 0.04 g/cc to about 0.11 g/cc

In some embodiments, patients being treated with methods described herein exhibit a reduction in the rate of disease progression as compared to patients not undergoing said pharmacotherapy. In some embodiments, said reduction in the rate of progression is at least 10%, or at least 20%, or at least 30%, or at least 50%, or at least 75% less than that rate of progression of said disease for patients not treated with the methods described herein.

In some embodiments, this disclosure provides a method for qualifying a patient for pharmacotherapy treatment of idiopathic pulmonary fibrosis with a composition as described herein, which method comprises:
a) selecting a patient diagnosed with idiopathic pulmonary fibrosis;
b) evaluating said patient to determine the stage of the disease for that patient;
c) disqualifying patients from said pharmacotherapy who are currently diagnosed as being in either Stage 3 or at Stage 4 for oxygen requirements of the disease;
d) initiating pharmacotherapy for patients who are determined to be at the incipient stage of the disease (Stage 1 of oxygen requirements) or at a moderate stage of the disease (Stage 2 of oxygen requirements);
e) monitoring said patients to determine the extent of progression of said disease after initiating pharmacotherapy; and
f) optionally removing said patients from pharmacotherapy for whom the attending clinician determines that pharmacotherapy is no longer effective.

In some embodiments, this disclosure provides for a method for treating qualified patients diagnosed with pulmonary fibrosis, which method comprises:
a) confirming that said patient has been qualified for said pharmacotherapy with a composition as described herein;
b) initiating pharmacotherapy for said patient;
c) maintaining said pharmacotherapy until the attending clinician determines that the disease progression has evolved such that there is no longer a benefit to the patient; and
d) terminating said pharmacotherapy.

In some embodiments, the attending clinician can ascertain the degree of progression of the disease based on the PF scale set forth herein. In some embodiments, the attending clinician can ascertain the degree of progression of the disease based on the increased rate of usage of oxygen.

Further contemplated are dry powder compositions of the disclosure comprising pirfenidone and/or nintedanib and any other therapeutic agents targeting PF or other pulmonary diseases, including but not limited to pulmonary arterial hypertension (PAH), and pulmonary hypertension in interstitial lung disease (PH-ILD and that can be administered by oral inhalation. PF-ILD is a serious, progressive lung condition where lung tissue thickens and scars (fibrosis or fibrosing), leading to worsening shortness of breath, cough, and reduced lung function. These symptoms impact a patient's quality of life and cause early mortality.

Accordingly, dry powder compositions of the disclosure comprising pirfenidone and/or nintedanib and treprostinil palmitil are contemplated. Treprostinil is currently in development for IPF and PPF. Treprostinil palmitil inhalation powder disclosed herein is a long-acting prodrug of treprostinil and a new chemical entity (NCE) that enables reduced daily dosing frequency compared to another form of treprostinil, which requires 4 doses daily. Exemplary doses of treprostinil palmitil are 80 to 640 microgram (mcg) doses administered once a day.

Additionally, dry powder compositions of the disclosure comprising pirfenidone and/or nintedanib and seralutinib are contemplated, which is an example of a triple drug combination. Seralutinib is a small-molecule drug inhibitor of platelet-derived growth factor receptor (PDGFR-alpha/beta), colony-stimulating factor 1 receptor (CSFIR), and mast or stem cell growth factor receptor kinases (SCFR). Seralutinib is being evaluated for the treatment of pulmonary arterial hypertension (PAH). Seralutinib can be administered by inhalation in twice daily doses, e.g., about 60 mg twice daily and up to about 90 mg twice daily.

It is further contemplated that compositions described herein may comprise any additional therapeutic and useful methods for treating PF or other pulmonary diseases and that can be administered by oral inhalation of the dry powder composition of the disclosure. Such methods comprise delivering via pulmonary inhalation an effective amount of the dry powder compositions as described herein. The methods can employ nintedanib and/or pirfenidone, and optionally a treprostinil, treprostinil palmitil, and/or seralutinib. The contemplated combinations can have 2, 3, 4, and even 5 APIs in the drug combination. Other contemplated therapeutics that can be used to treat a PF patient can include admilparant, buloxibutid, nerandomilast, tadalegib, vismodegib, or ENV-101. These can be administered separately instead of as a fixed dose combination of medications, as for example a fixed dose combination of nintedanib and pirfenidone.

In some contemplated embodiments, the methods can employ pirfenidone, nintedanib, and treprostinil palmitil powder. In some contemplated embodiments, the methods can employ pirfenidone and treprostinil palmitil. In some contemplated embodiments, the methods can employ nintedanib and treprostinil palmitil. In some contemplated embodiments, the methods can employ pirfenidone and seralutinib. In some contemplated embodiments, the methods can employ pirfenidone, treprostinil palmitil, and/or seralutinib. In some contemplated embodiments, a dry powder composition comprising both nintedanib and pirfenidone and one or both of treprostinil palmitil and seralutinib is used to treat PF or other pulmonary diseases in a patient. It is contemplated that when so used, the combination of the three or four drugs will benefit the patient because the drugs have unique mechanisms of action relative to the other drugs. It is contemplated that such unique action by each drug may be synergistic.

### EXAMPLES

In the following examples, the following terms are used and have the following meanings. Terms not defined have their scientifically accepted meanings.

| | | |
|---|---|---|
| calcium chloride | = | anhydrous calcium chloride |
| DPI | = | dry powder inhaler |
| mg | = | milligram |
| mL | = | milliliter |
| Temperature | = | all temperatures reported are +/− 15% of |

Nintedanib or nintedanib esylate as used in the following examples refers to the compound represented by formula II or II-A, wherein R is methyl. The following examples indicate whether they are exemplary, which reflect prophetic details for use with the embodiments described herein.

### Exemplary Examples 1-5-Formation of Emulsion Containing Varying Weight Percent Solids

A stable nanoemulsion is first formulated for subsequent use in dry spraying. A nanoemulsion is prepared by combining the components of Table 3 as follows:

Add the following together:
a) ultrapure water, heated to above 60° C.; and
b) distearoylphosphatidylcholine (DSPC)
   The lipid, e.g., DSPC, must be dispersed at a temperature above its Tm, above about 55° C. Dispersion is obtained using a high-shear mixer as a means of admixing the two components.

High-shear mixing is performed using standard conditions (10,000 rpm) for about 5 minutes (e.g., with a Turrax high-shear mixer). Calcium chloride is added to the composition, and admixing is continued for about 5 minutes (about 10,000 rpm) thereby forming a plurality of DSPC liposomes in solution. Add the suspension medium (SM) as described in U.S. Pat. No. 9,554,993 (col. 37-38), the content of the patent is incorporated herein by reference in its entirety, while stirring the composition at about 15,000 rpm. This coarse emulsion is then processed in a high-pressure homogenizer to form a fine emulsion. The composition is removed and can be stored until used in the next step.

Under high-shear mixing, add to the emulsified composition the requisite amounts of both nintedanib esylate (N) and pirfenidone (P), either separately or together, at the desired ratio of these two actives and in an amount to achieve the desired amounts of solids to be used. If necessary, a further homogenization step can be used to reduce the size of the drug particles.

Table 3 provides exemplary formulation examples of suitable emulsified formulations, where the suspending medium (SM) is PFOB:

TABLE 3

| | Example | Example | Example | Example | Example |
|---|---|---|---|---|---|
| | 1 | 2 | 3 | 4 | 5 |
| | 3% | 1% | 1.5% | 2% | 2.5% |
| Component | solids | solids | solids | solids | solids |
| Water | 170 mL | 185 mL | 175 mL | 180 mL | 170 mL |
| SM | 30 mL | 15 mL | 25 mL | 20 mL | 30 mL |
| DSPC | 1,230 mg | 410 mg | 615 mg | 820 mg | 1,025 mg |
| CaCl₂ | 120 mg | 40 mg | 60 mg | 80 mg | 100 mg |
| N | 150 mg | 40 mg | 75 mg | 100 mg | 125 mg |
| P | 1,500 mg | 510 mg | 750 mg | 1,000 mg | 1,250 mg |
| Total | 3,000 mg | 1,000 mg | 1,500 mg | 2,000 mg | 2,500 mg |
| solids | | | | | |

### Exemplary Examples 6-10-Formation of a Dry Powder

Spray drying is a process used to convert an atomized liquid feedstock into a dry powder.

In each of Exemplary Examples 1 to 5, each feedstock can be spray dried using a custom-built, laboratory-scale spray dryer comparable in scale to a commercially available Büchi 191 mini spray dryer. A twin-fluid atomizer can be employed to generate droplets. The droplet size is governed by the air-to-liquid ratio (ALR) within the atomizer. The atomized droplets are dried through contact with a heated gas stream. The air stream can be air or nitrogen. The resulting particles can be collected using a cyclone separator.

### Example 11—Exemplary Dosing Ranges

An exemplary total daily dose of pirfenidone can be 20 mg to 80 mg for direct pulmonary delivery. An exemplary total daily dose of nintedanib can be 0.4 mg to 1.6 mg for direct pulmonary delivery. Exemplary total daily dose of a treprostinil (T) (free form) can be 0.07 mg to 0.3 mg for direct pulmonary delivery.

Dosing ranges for a dry powder composition containing an effective amount of pirfenidone (P) and of nintedanib (N) can range from a weight ratio of about 8:1 (P: N) to about 40:1. Exemplary weight ratios include: 15:1, 20:1, 25:1, 30:1, and 40:1 (and any value between 8 and 40).

Dosing ranges for a dry powder composition containing an effective amount of pirfenidone (P) and of a treprostinil (T) can range from a weight ratio of about 60:1 (P: T) to about 1,200:1. Exemplary weight ratios include: 65:1, 100:1, 300:1, 500:1, and 1,150:1.

Dosing ranges for a dry powder composition containing an effective amount of nintedanib (N) and of a treprostinil (T) can range from a weight ratio of 3:1 (N: T) to about 6:1. Exemplary weight ratios include: 4:1, 5.3:1, and 5.7:1.

Dosing ranges for a dry powder composition containing an effective amount of pirfenidone (P), nintedanib (N) and of a treprostinil (T) can range from a weight ratio of 65:5:1 (P:N:T), to about 100:4:1 (P:N:T) or to about 1200:6:1. Exemplary weight ratios include, but are not limited to, 67:5:2, 100:4:1, 265:5:1, 290:6:1, 400:4:1, and 1, 150:6:1.

### Example 12—Exemplary Dosing Ranges

An exemplary total daily dose of pirfenidone can be about 20 mg to about 80 mg for direct pulmonary delivery. An exemplary total daily dose of nintedanib can be about 0.4 mg to about 1.6 mg for direct pulmonary delivery. Exemplary total daily dose of treprostinil palmitil can be 0.08 mg to 0.64 mg for direct pulmonary delivery.

Dosing ranges for a dry powder composition containing an effective amount of pirfenidone (P) and of nintedanib (N) can range from a weight ratio of about 10:1 (P:N) to about 250:1. Exemplary weight ratios include but are not limited to: 12.5:1, 25:1, 50:1, 100:1, and 200:1.

Dosing ranges for a dry powder composition containing an effective amount of pirfenidone (P) and of treprostinil palmitil can range from a weight ratio of about 30:1 (P: treprostinil palmitil) to about 1000:1. Exemplary weight ratios include: 90:1, 1251, 250:1, and 350:1.

Dosing ranges for a dry powder composition containing an effective amount of nintedanib (N) and treprostinil palmitil can range from a weight ratio of 2:1 (N: treprostinil palmitil) to about 10:1. Exemplary weight ratios include: 2.5:1, 3.5:1, 4:1, and 5:1.

Dosing ranges for a dry powder composition containing an effective amount of a triple drug combination comprising pirfenidone (P), nintedanib (N) and treprostinil palmitil can range from a weight ratio of 30:3:1 (P: N: treprostinil palmitil), to about 350:3.5:1 (P:N: treprostinil palmitil) or to about 1000:5:1. Exemplary weight ratios include, but are not limited to, for example 90:3.5:1, 125:3:1, 250:5:1, and 350:3.6:1.

### Example 13

In this example, solution-based formulations (Formulations PSOL-1 through PSOL-8) were based upon spray-drying a feedstock consisting of a single-phase, homogeneous aqueous solution. Such a feedstock was prepared by dissolving drug(s) and excipients in water.

As a specific example, the following approach was used to prepare the feedstock for formulation PSOL-7. About 200 mL of HPLC-grade water was added to a 250 mL volumetric flask on a stir plate at room temperature. Excipients (leucine and anhydrous calcium chloride) were added as indicated below, one at a time, to the flask while stirring using a magnetic stir bar. Then, nintedanib esylate was added, followed by pirfenidone.
2800.03 mg of Leucine
495.00 mg of CaCl₂) (anhydrous)
55.06 mg of Nintedanib esylate
1653.31 mg of Pirfenidone

The mixture was continuously stirred until a clear solution was obtained. The stir bar was then removed. HPLC-grade water was added to bring the total volume to 250 mL. After confirming that the pH was within the range 5.9±0.3. The solution was filtered through a 0.22 μm membrane, which is either a PES (polyethersulfone) or PVDF (polyvinylidene fluoride) membrane.

### Example 14

Solution-based porous particle formulations (Formulations PSPH-1 through PSPH-3) are based upon spray-drying an emulsion-based feedstock. In this example, the drugs were dissolved in a continuous phase (water) of the oil-in-water emulsion.

The oil-in-water emulsion comprised PFOB droplets, a suspension medium, stabilized by a monolayer of distearoylphosphatidylcholine (DSPC) molecules and calcium ions. A lipid dispersion was prepared by mixing the required quantities of calcium chloride dihydrate and DSPC into HPLC-grade water and heated to a temperature of 60-80° C. The elevated temperatures ensure that the acyl chains in the long-chain phospholipid are present in the disordered ‘liquid crystalline’ orientation (i.e., above the main transition temperature of DSPC, 55° C.). The materials were mixed at 10,000 rpm for at least 5 minutes with an IKA T-25 UltraTurrax disperser equipped with a S25N-10G dispersing element to form a dispersion of multilamellar vesicles. The PFOB was then metered dropwise into the dispersion under high-shear mixing to form a coarse emulsion. The coarse emulsion was mixed for an additional 10 min at 10,000 rpm. The coarse emulsion was cooled to room temperature and then passed through an Avestin Emulsiflex C-5 high pressure homogenizer at a pressure of 10 kpsi for a period of 10 min, followed by a single discrete pass at 15-20 kpsi. The heat exchanger of the homogenizer was cooled with using a Lauda RK8CS circulating bath at a temperature of 5° C. The concentrated fine emulsion was then set aside. Separately, a concentrated drug annex solution was prepared by dissolving each drug, one at a time, in HPLC-grade water. The drug solution was filtered through a 0.22 μm PES or PVDF membrane.

The feedstock was prepared by adding the drug annex solution to the fine emulsion, followed by addition of HPLC-grade water to attain the target total volume. To ensure homogeneity, the feedstock was continuously mixed during spray drying.

A summary of the primary particle sizes of selected spray-dried powders is shown in Table 4 below.

TABLE 4

| Sample | Dx (10) (μm) | Dx (50) (μm) | Dx (90) (μm) |
|---|---|---|---|
| PSOL-6 | 0.882 | 1.74 | 12.8 |
| PSPH-1 | 0.729 | 1.52 | 4.48 |
| PSPH-2 | 0.680 | 2.36 | 5.92 |
| PSPH-3 | 0.657 | 1.86 | 7.02 |

### Example 15 (Prophetic)

Suspension-based porous particle formulations were based upon spray-drying an emulsion-based feedstock that contained suspended crystalline drug particles. This approach is useful for solid-state forms (salts, polymorphs, etc.) that have poor aqueous solubility. Maintaining the drug in the crystalline state can result in a spray-dried powder with improved chemical stability. In this approach, one or more of the drugs can be suspended in the continuous phase (water) of the oil-in-water emulsion. Thus, the feedstock comprises an emulsion-suspension with at least two discrete phases: oil droplets and one or more drugs present as suspended crystalline particles.

In this example, the oil-in-water emulsion comprised PFOB droplets stabilized by a monolayer of distearoylphosphatidylcholine (DSPC) molecules and calcium ions. A concentrated emulsion was prepared by mixing the required quantities of calcium chloride dihydrate and DSPC into HPLC-grade water and heated to a temperature of about 60-80° C. The use of the elevated temperatures ensured that the acyl chains in the long-chain phospholipid are present in the disordered ‘liquid crystalline’ orientation (i.e., above the main transition temperature of DSPC, 55° C.). The materials were mixed at 10,000 rpm for at least 5 minutes with an IKA T-25 UltraTurrax disperser equipped with a S25N-10G dispersing element to form a dispersion of multilamellar vesicles. The PFOB was then metered dropwise into the dispersion under high-shear mixing to form a coarse emulsion. The emulsion was mixed for an additional 10 min at 10,000 rpm. The coarse emulsion was cooled to room temperature and then passed through an Avestin Emulsiflex C-5 high pressure homogenizer at a pressure of 10 kpsi for a period of 10 min, followed by a single discrete pass at about 15-20 kpsi. The heat exchanger of the homogenizer was cooled using a Lauda RK8CS circulating bath at a temperature of about 5° C.

If a drug has sufficient aqueous solubility, it can be added directly to the emulsion, as seen in Example 14. Alternatively, a drug can be prepared as a separate drug annex solution by dissolving the drug in water and then adding this solution to the emulsion.

If a drug has poor aqueous solubility, the powdered drug is added to the fine emulsion while mixing with the Ultra Turrax (~20,000 rpm for about 10-30 min, until the drug is fully incorporated; the use of “~” is equivalent to using the term “about”). In some cases, this may require swirling of the vessel to wet the powder. The swirling drives the powder on the surface of the liquid beneath the surface to interact with the emulsion droplets. To reduce the size of the drug particles or to promote more intimate mixing of the drug and the emulsion, the mixture can be homogenized. To homogenize the resulting mixture, the emulsion-suspension is passed through the high-pressure homogenizer for a period of about 10 min, followed by a single discrete pass at about 15-20 kpsi. The weight of the concentrated emulsion is then compensated for water evaporation and diluted to a target concentration. The resulting feedstock can have a milky appearance, with any flocs of drug well dispersed within the continuous aqueous phase. To ensure homogeneity, the suspension-based feedstock is continuously mixed during spray drying.

### Example 16—Spray-Drying

Spray drying is a process used to convert an atomized liquid feedstock into a dry powder.

In this example, each feedstock was spray dried using a custom-built, laboratory-scale spray dryer comparable in scale to a commercially available Büchi 191 mini spray dryer. A twin-fluid atomizer was employed to generate droplets. The droplet size was governed by the air-to-liquid ratio (ALR) within the atomizer. The atomized droplets were dried through contact with a heated gas stream. The air stream can be air or nitrogen, and the resulting particles were collected using a cyclone separator. The target spray-drying conditions are summarized in Table 5.

TABLE 5
Spray-drying process parameters used for powder manufacturing

| Parameter | Target |
|---|---|
| Atomizer Liquid Feed [mL/min] | 2.3 |
| Dry Gas Flow Rate [L/min] | 600 |
| Atomizer Gas Flow Rate [L/min] | 21.5-25.0 |
| Atomizer Gas Pressure [kPa] | 345 |
| Inlet Temperature [° C.] | 80-98 |
| Outlet Temperature [° C.] | 65-72 |

### Example 17

HPLC analysis of the spray-dried powders indicated that recovery of pirfenidone was frequently less than 100%. Care was taken to confirm that this loss was not attributable to errors in feedstock preparation. Recovery of pirfenidone as a function of spray-dryer outlet temperature is shown in FIG. 2. These data indicate that the spray dryer outlet temperature is a critical process parameter for pirfenidone recovery, with higher outlet temperatures resulting in reduced recovery.

A second feature of FIG. 2 is that the individual data points are labeled according to the mass ratio of divalent cation to pirfenidone. Examination of the data suggests that divalent cations improve pirfenidone recovery. Although the mechanism underlying this improvement is not known, one hypothesis is that it is related to chelation of metal ions by pirfenidone. Such chelation could reduce the vapor pressure of pirfenidone, thereby decreasing its sublimation rate (see discussion below) and improving recovery. An alternative hypothesis relates to pirfenidone's structural isomerism, specifically keto-enol tautomerism. It is possible that coordination with metal ions shifts this equilibrium, stabilizing pirfenidone in a form less prone to sublimation.

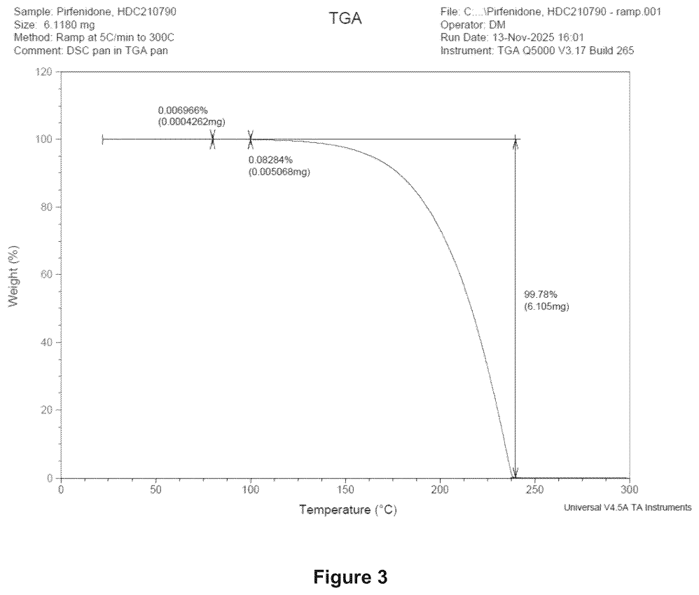

Further work was done to understand the mechanism of pirfenidone loss during spray drying. FIG. 3 shows the TGA thermogram of pirfenidone heated at 5° C./min. These data are of particular interest given the poor recovery of pirfenidone in some of the spray-dried powders. Mass loss values at temperatures representative of spray-drying outlet conditions (e.g., about 80° C. and about 100° C.) are annotated; these values are 0.007% and 0.08%, respectively. The sublimation rate is expected to depend strongly on the specific surface area of the particles. Therefore, the sublimation rate of micron-sized spray-dried pirfenidone particles would likely be significantly higher than that of the bulk drug substance.

The TGA thermogram of pirfenidone shows that the mass loss rate gradually increases as temperature rises, with the sample eventually undergoing complete mass loss by about ~240° C. This behavior is consistent with sublimation.

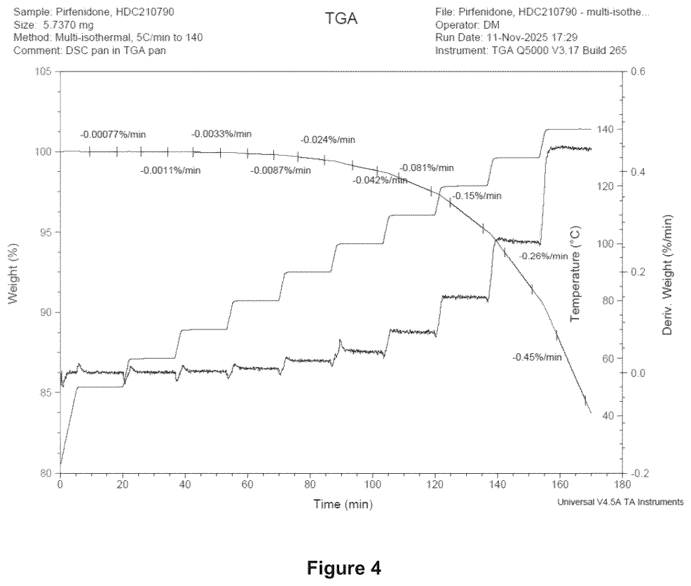

To investigate this further, another TGA experiment was performed in which pirfenidone was heated in 10° C. increments from 50 to 140° C., holding isothermally for 15 min at each temperature. FIG. 4 shows the average mass-loss rate at each step. The derivative mass loss trace is also shown. These data indicate that mass loss rate increases markedly above ~100° C., which is near pirfenidone's melting point (108° C.). Strictly speaking, sublimation is a solid-vapor phase transition and would occur below the melting point of pirfenidone. Mass loss above the melting point is a liquid-vapor phase transition and is due to evaporation of the (liquid) melt. In either the case of solid or liquid pirfenidone, it has a non-negligible vapor pressure and will either sublime or evaporate.

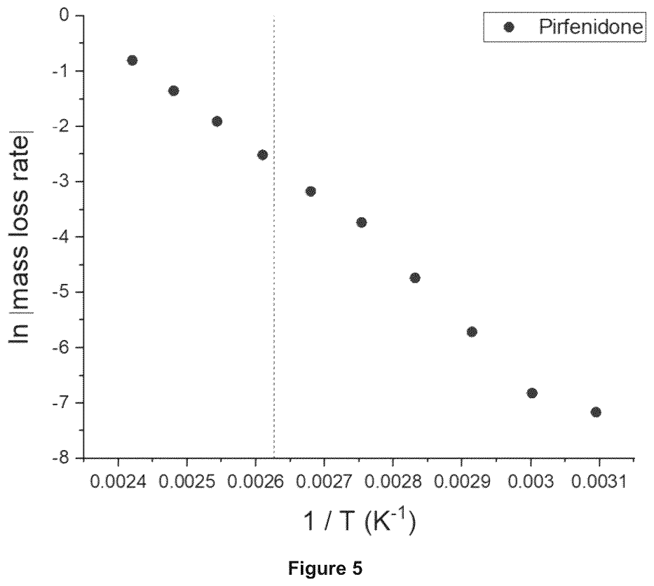

The mass loss rates from the stepwise isothermal TGA experiment were plotted in an Arrhenius plot (FIG. 5). The vertical dotted line in FIG. 5 represents the melting temperature of pirfenidone expressed in inverse Kelvin units. These data are approximately linear across the Tₘ, indicating that the sublimation and evaporation processes below and above Tₘ, respectively, have similar kinetics.

Visual evidence of sublimation during thermogravimetry was also observed. After the experiment, a fine white deposit was visible on the bottom surface of the heat exchange baffle, consistent with condensed pirfenidone vapor. This occurs when pirfenidone vapor rises and deposits on nearby, cooler surfaces. This was also observed following spray drying, where elevated levels of pirfenidone were detected on the cap of the cyclone. The sublimation rate is expected to depend strongly on the specific surface area of the particles. Therefore, the sublimation rate of micron-sized spray-dried pirfenidone particles would likely be significantly higher than that of the bulk drug substance.

### Example 18

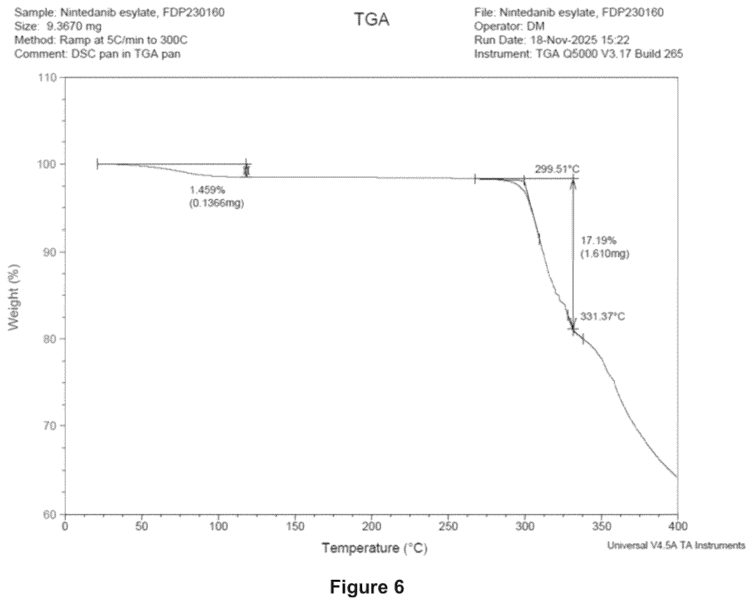

FIG. 6 shows the TGA thermogram of nintedanib ethanesulfonate (alternatively, nintedanib esylate, NE). When heated at 5° C./min, the sample exhibits a gradual mass loss of approximately 1.46% w/w. Based on the proximity of the end of this event (ending just above 100° C.) to the boiling point of water, the mass loss is attributed to the removal of water from the crystalline hydrate. On a molar basis, 1.5% corresponds to 0.5 mol of water per mole of NE, consistent with an NE hemihydrate form. This hydrated form has been previously reported in the literature (e.g., Liu C, Zhang H, Li Y, Wang J. “Solubility measurement and thermodynamic properties of nintedanib esylate hemihydrate in pure solvents,” J. of Molecular Liquids. 2023; 390:122900). Upon further heating, a larger mass loss was noted above about 300° C. due to gross thermal decomposition of the melt.

The first and second heating scans obtained by a differential scanning calorimetry measurement (DSC) on NE are shown in 7. A pinholed pan was used to allow water to escape from the sample during heating. The first DSC scan shows a broad endotherm from approximately 25 to 125° C. This diffuse endotherm is consistent with the gradual water loss observed in the TGA experiment. The DSC thermogram also shows a sharp endotherm with an onset of 125-130° C.

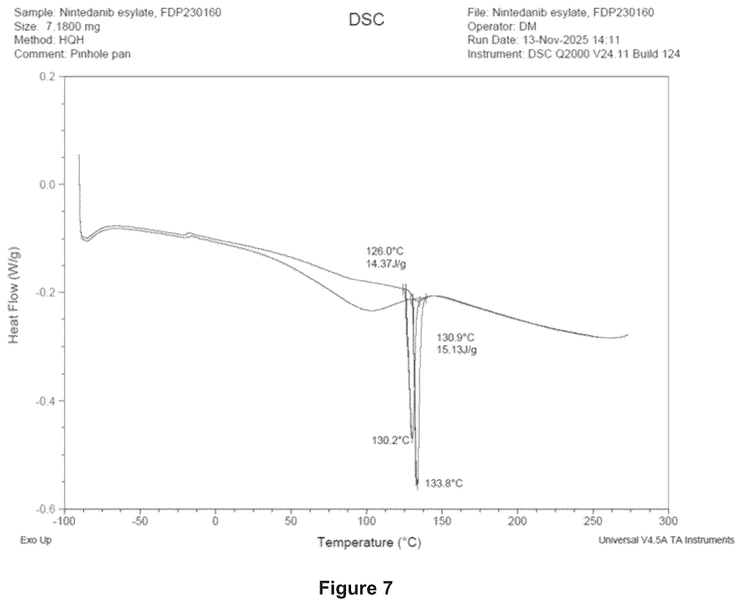

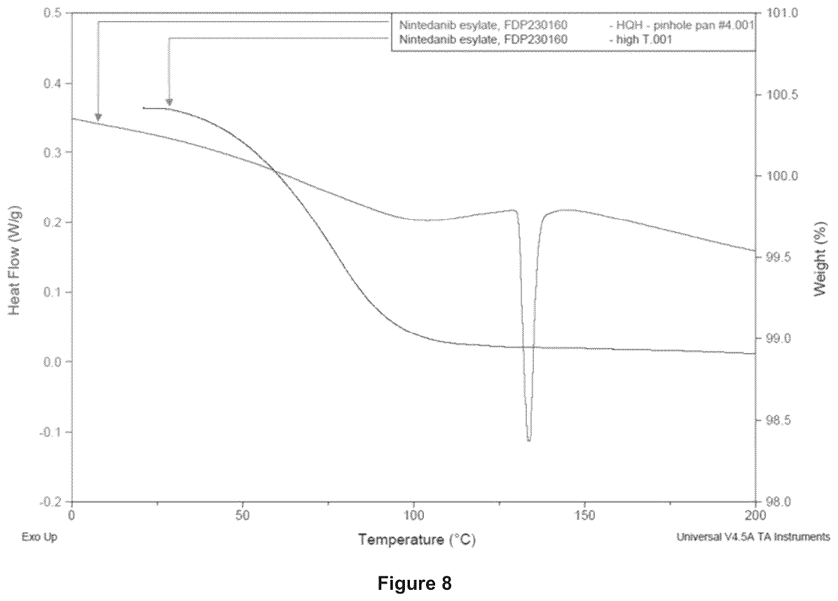

Because this diffuse endotherm appears in both the first and second heating cycles (after the material is fully dehydrated), it could represent a polymorphic transition between two anhydrous forms (one created by dehydration of the hemihydrate and another from a polymorphic solid-solid transition during heating in the DSC). This is supported by TGA data showing that this transition is not associated with a change in sample mass (FIG. 8). Note that, in this first experiment (FIG. 7), the sample was heated to 255° C., which was not sufficient to melt the anhydrous form.

A heat-quench-heat experiment is conducted to understand the glass transition and recrystallization behavior of a given material. This type of DSC experiment has three steps. The objective of the first heating scan is to measure the melting temperature and any other thermal events of the as-received sample. After heating to a temperature just above the melting point, the sample is quenched and cooled to −90° C. within the DSC using a ‘jump’ step. The purpose of this quenching step is to rapidly supercool the melted material before crystallization can occur. The rapid quenching favors the formation of an amorphous solid (glass). The purpose of the second heating scan is to measure the glass transition temperature (Tg) of the melt-quenched sample. This approach provides a reasonable estimate of Tg, although small amounts of impurities generated during melting could influence Tg.

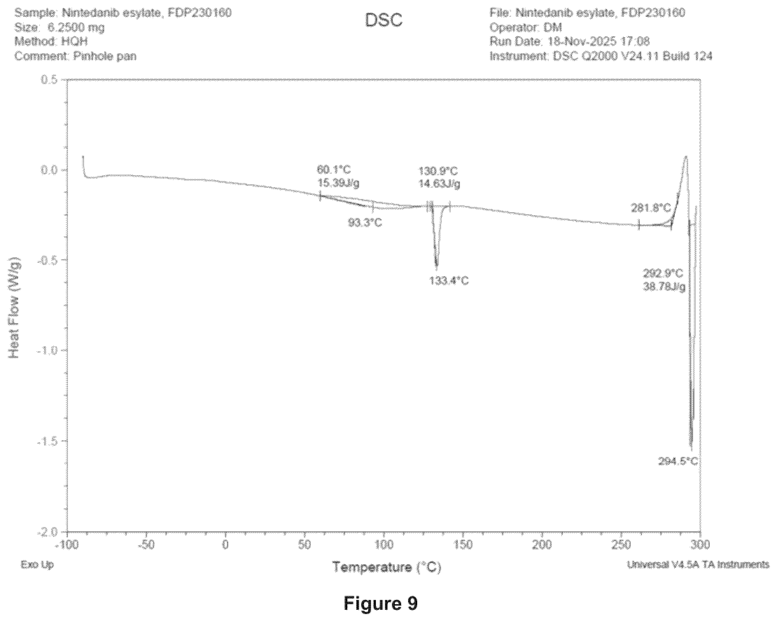

In a heat-quench-heat experiment on the nintedanib esylate (NE), the NE sample was heated to nearly 300° C. during the first heating scan. To avoid excessive degradation of the melt (as observed in the TGA data), higher temperatures were not used. This first heating scan (FIG. 9) showed the same thermal events seen in the previous experiment: a broad endotherm followed by a sharp endotherm. Additionally, a high-temperature melting endotherm was observed with an onset near 300° C., indicating that the melting point of anhydrous NE is unusually high compared with most organic crystalline drugs.

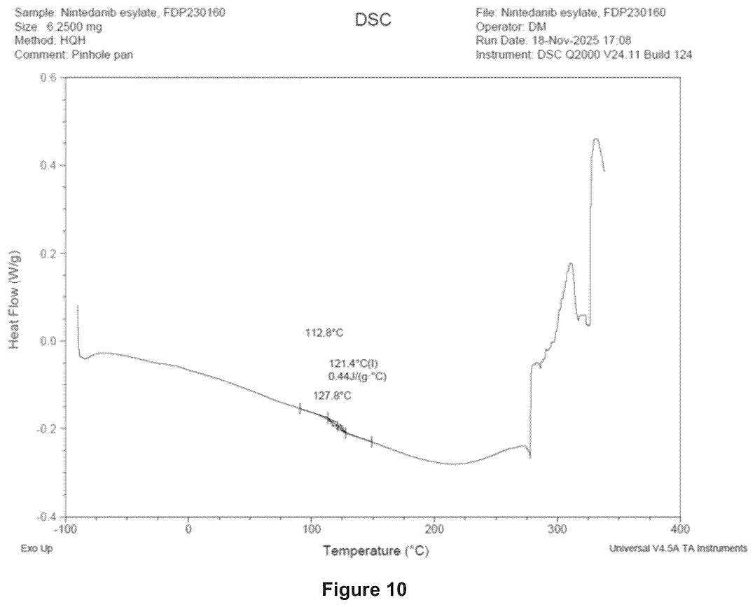

The second heating scan shows a glass transition at approximately 113° C. (FIG. 10). No recrystallization exotherm is observed upon further heating.

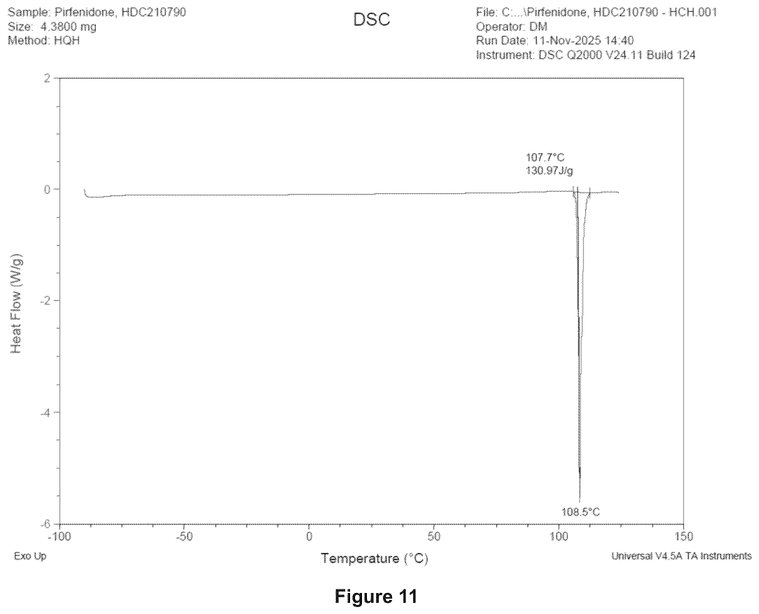

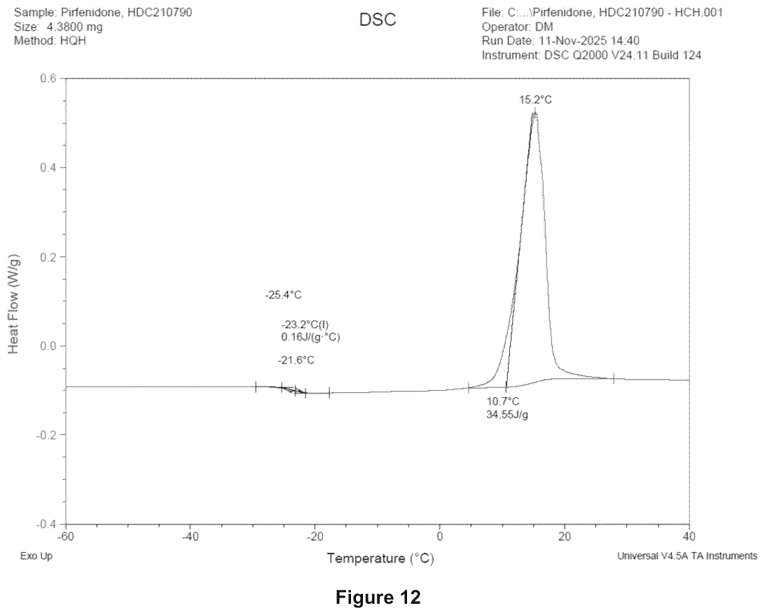

The first-heating DSC scan of pirfenidone is shown in FIG. 11. The material exhibits a melting temperature of 107.7° C., which is consistent with the literature values (102-111° C.) (Kumari, N., Bhattacharya, B., Roy, P., Michalchuk, A. A. L., Emmerling, F., and Ghosh, A., “Enhancing the Pharmaceutical Properties of Pirfenidone by Mechanochemical Cocrystallization,” Crystal Growth & Design, 19(11): 6482-6492, 2019). The second heating scan (FIG. 12) indicates a glass transition at −25° C., followed by recrystallization at approximately 11° C.

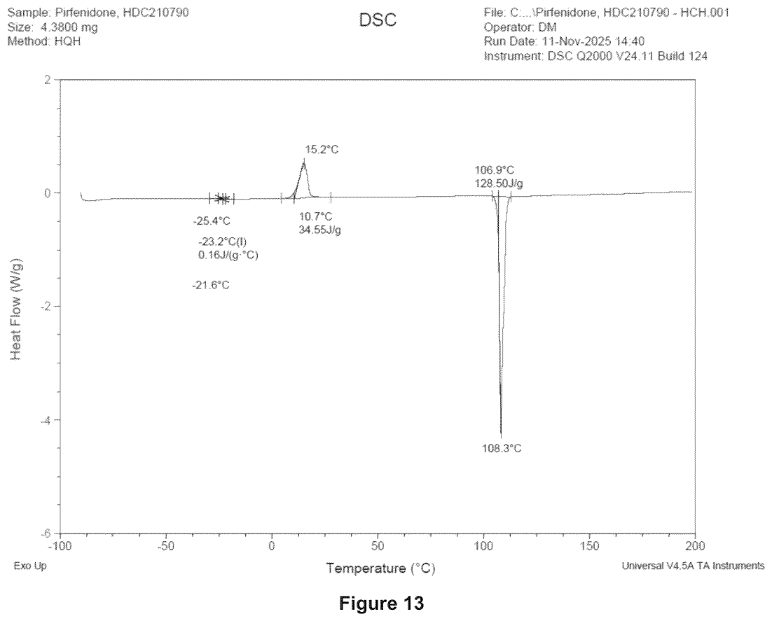

Upon further heating, the recrystallized material melted at ~107° C. (FIG. 13). These data indicates that pirfenidone readily recrystallizes over the time scale of the DSC experiment.

Two factors promote this behavior: (1) the sample's low melting temperature, which allows melting without significant thermal degradation, and (2) its low molecular weight (185 g/mol) and limited number of rotatable bonds, both of which favor crystallization.

The glass transition temperature of pirfenidone is quite low. At ambient temperature, amorphous pirfenidone would be above its Tg and is considered to be a viscous liquid. Given the proximity of this Tg to pharmaceutical storage temperatures (e.g., about 25° C. and 40° C.), amorphous pirfenidone-if present-could create challenges with stability of the drug substance. Although the drug might be chemically stable, the molecular mobility during storage near or above Tg could cause physical changes to the drug (viscous flow, sintering, recrystallization).

As quench-cooled pirfenidone is heated through its Tg, the material transforms from a mechanical solid to a viscous, supercooled liquid. As the sample is heated further, the viscosity decreases, and recrystallization can occur over the timescale of the DSC experiment at any temperature between T_{g} (−25° C.) and Tₘ (≈108° C.). Recrystallization typically occurs at temperatures more than 15-20° C. above T_{g}, when the viscosity of the supercooled liquid is high enough to enable crystal nucleation and growth on the timescale of the DSC experiment. Depending on the time-temperature history of particles during spray drying, in-process recrystallization of pirfenidone might be possible.

Table 6 shows the measured melting temperatures and the glass transition temperatures determined by melt quenching.

The glass transition temperature can also be estimated using an empirical rule based on the melting temperature, Tₘ. For example, based on a linear regression of measured T_{g} and Tₘ values of 70 different amorphous pharmaceuticals (see Kerc, J. and Srcic, S., “Thermal analysis of glassy pharmaceuticals,” Thermochim. Acta 248:81-95, 1995), the T_{g}/Tₘ was found to be 0.73±0.10. The calculated T_{g}/Tₘ values of nintedanib esylate, nintedanib free base, and pirfenidone are within the range of other amorphous, organic small molecules.

TABLE 6
Measured melting temperature and glass transition
temperature determined by melt quenching.

| Molecule | Tₘ (° C.) | T_{g} [melt quenched] (° C.) | T_{g}/Tₘ |
|---|---|---|---|
| nintedanib esylate | 292.9 | 112.8 | 0.68 |
| nintedanib free base | 240.5 | 75.6 | 0.68 |
| pirfenidone | 107.7 | −25.4 | 0.65 |

### Example 19—X-Ray Powder Diffraction (XRPD)

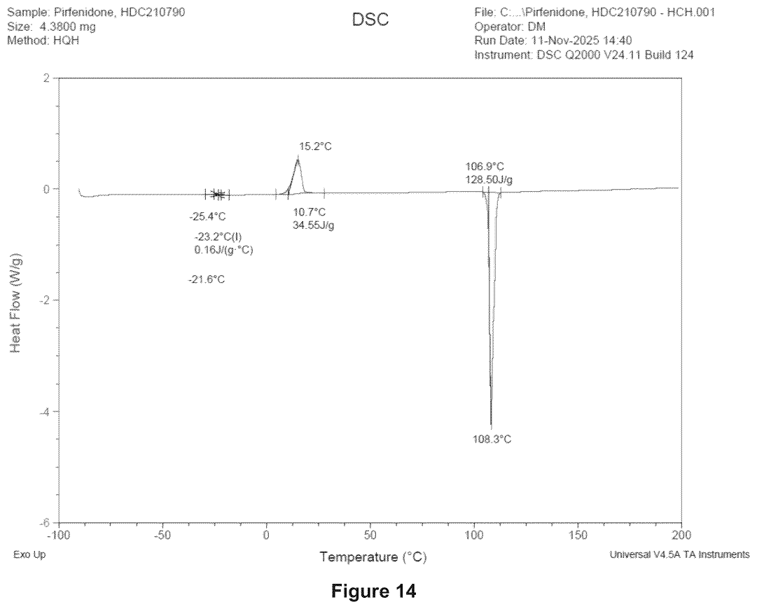

An overlay of the PXRD patterns of nintedanib esylate and pirfenidone is shown in FIG. 14.

### Example 20—XRPD

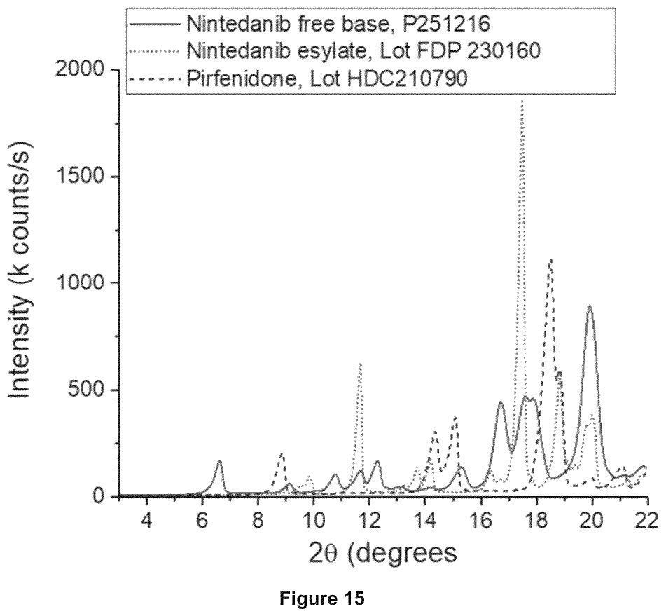

One exemplary fixed-dose combination comprises pirfenidone and nintedanib esylate. Thus, it is of interest to assess whether there are diffraction peaks that uniquely identify each form in a formulation that contains both drugs. An overlay of the low-angle region of the powder patterns of nintedanib esylate, nintedanib free base, and pirfenidone is shown in FIG. 15. For a fixed-dose combination of pirfenidone and nintedanib esylate, there are low-angle peaks below 15°2θ that uniquely identify each crystal: pirfenidone (e.g., 8.8 and 15.1°2θ), nintedanib esylate (e.g., 9.9, 11.7, and 17.4°2θ).

The as-received nintedanib esylate DS was overlayed and compared with the powder patterns of polymorphs discussed in U.S. Pat. No. 7,119,093 B2 (assigned to Boehringer Ingelheim) and in another patent filed by Suven Life Sciences (India), WO 2016/178064A1. Although the solid-state form of the as-received nintedanib esylate DS is not consistent with the polymorph reported in U.S. Pat. No. 7,119,093, the solid-state form of nintedanib esylate DS is consistent with the “Form S”, as designated by Suven Life Sciences. The pattern for the “Form S” is also consistent with that reported by Ma et al. (see, Ma, J., Huang, J., Cao, Z., Sha, J., Sun, R., He, H., Wan, Y., Li, Y., Li, T., and Ren, B., “Solubility measurement and thermodynamic properties of Nintedanib Esylate Hemihydrate in pure solvents,” J. Molecular Liquids 352:118624, 2022). The material used in the Ma et al. (2022) study is indicated as having been purchased from Shanxi Didu Pharmaceutical Chemical Co. LTD (China).

### Example 21—Preparation of Nintedanib Free Base

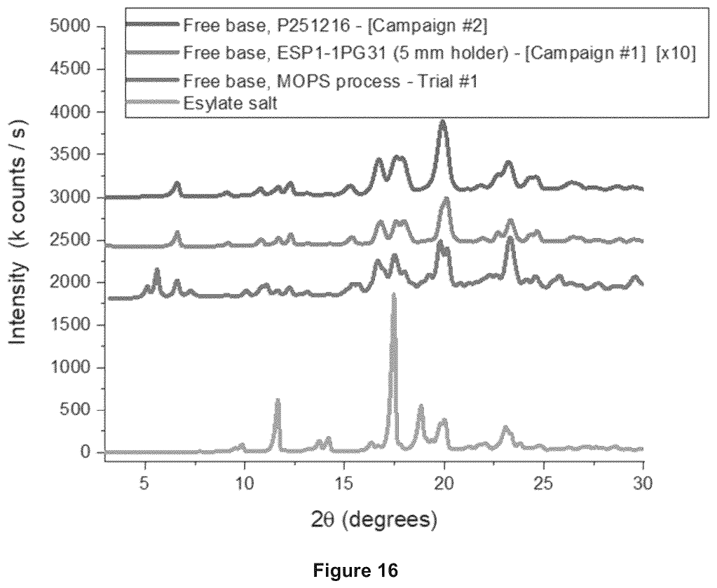

Using nintedanib esylate as the starting material, nintedanib free base was prepared by deliberate disproportionation. FIG. 16 shows an overlay of the X-ray powder diffraction data of the different preparations of nintedanib free base. The legend shows the chronological order of batches, with the batch size increasing over time, with batch sizes increasing from about 5 to about 15 grams, with yields of about 50-70%. The first preparation showed additional peaks at about 5.1, 5.6, 7.3, and 10.1°20, possibly resulting from residual buffer salts.

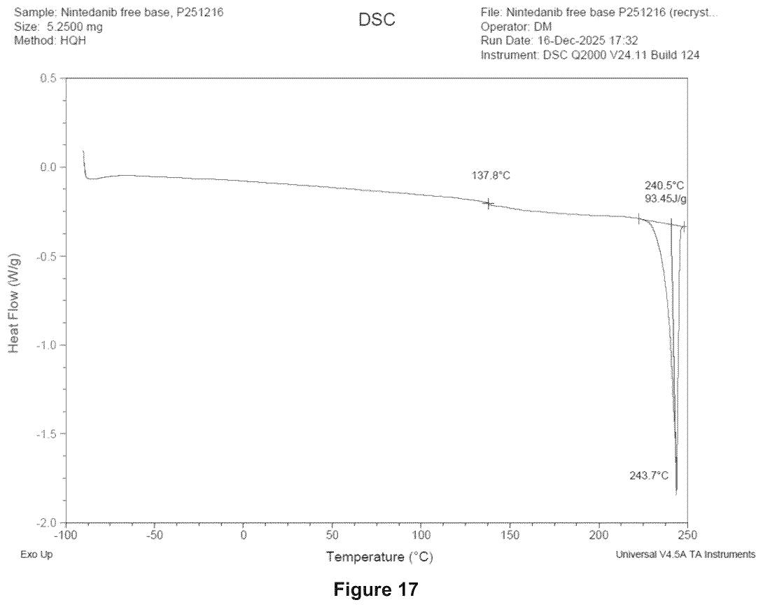

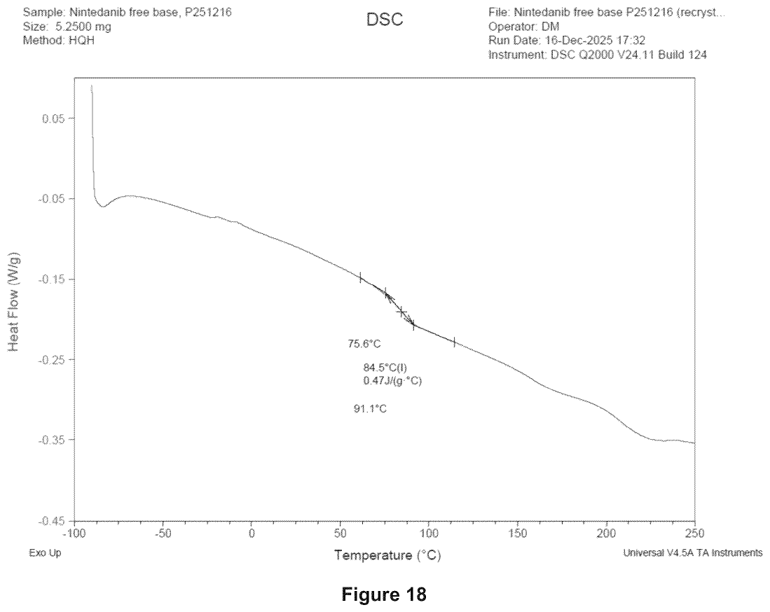

The first and second DSC heating scans on nintedanib free base are shown in FIG. 17 and FIG. 18, respectively. The first heating scan shows a sharp melting peak occurring at 241° C. (ΔH=93.5 J/g). As expected, the melting temperature of the free base is lower than that of the salt (esylate) form (293° C.). However, both forms have high melting temperatures.

After quench cooling in the DSC, the second heating scan (FIG. 18) shows a glass transition with an onset at 76° C. No recrystallization or melting was observed.

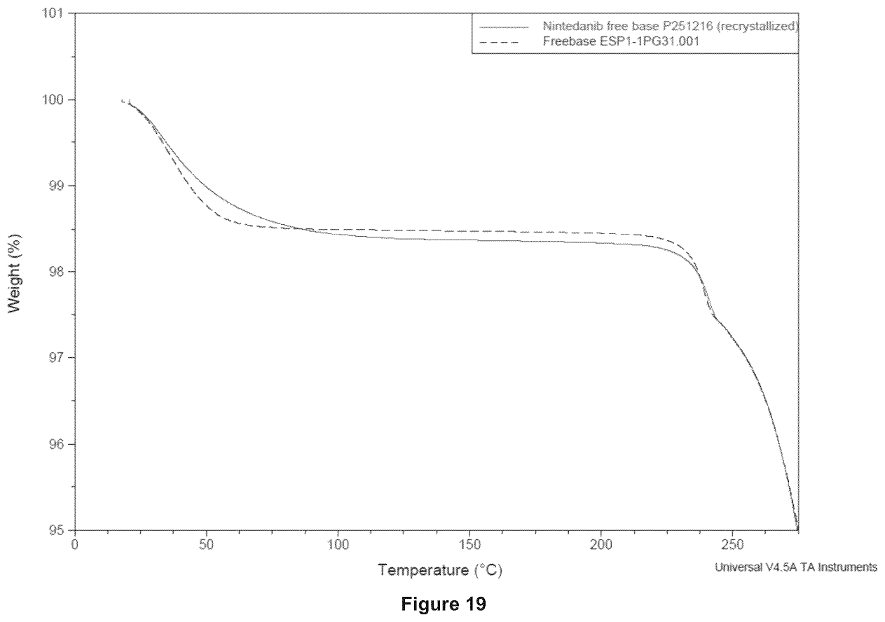

When using nintedanib free base for formulation work, it is important to correct for residual solvent content (loss on drying). FIG. 19 shows an overlay of the TGA thermograms of the preparations of nintedanib free base used in spray-drying Campaign #1 (ESP1-1PG31) and Campaign #2 (P251216). Both campaigns exhibit similar loss on drying of about 1.5 to 1.6% w/w. Although stoichiometrically this loss on drying corresponds to nintedanib free base hemihydrate, it is not known whether this material is a stoichiometric hydrate or the composition corresponds to water associated with amorphous material.

Both recrystallized batches also exhibit similar time-temperature profiles, providing qualitative information on the comparability of these materials. A significant mass loss occurs above about 240° C. due to thermal decomposition during and after melting.

It is contemplated that nintedanib free base may be preferred in manufacturing a dry powder composition described herein. Using nintedanib free base may provide the following advantages: (i) its crystalline form may offer improved stability relative to the amorphous form; (ii) its lower solubility could contribute to longer residence time in the lungs; and (iii) avoidance of disproportionation may improve tolerability, both in the oropharynx (less cough, less dysgeusia) and in the lungs (no acid released on the lung epithelium from disproportionation of esylate salt to form ethanesulfonic acid).

### EMBODIMENTS

The following embodiments are exemplary embodiments of the dry powder compositions, methods of making the dry powder compositions and methods of using the compositions and should not be viewed as limiting.

Embodiment 1A. A dry powder composition comprising an effective amount of a) pirfenidone, and b) nintedanib or a pharmaceutically acceptable salt thereof, and which further comprises c) a di-fatty acid phosphatidyl choline, and d) calcium chloride.

Embodiment 2A. The dry powder composition of Embodiment [1A], wherein said pharmaceutically acceptable salt of nintedanib is nintedanib esylate.

Embodiment 3A. The dry powder composition of either Embodiment [1A] or Embodiment [2A], wherein said pirfenidone, and nintedanib or a pharmaceutically acceptable salt thereof are uniformly dispersed in the dry powder composition.

Embodiment 4A. The dry powder composition of any one of Embodiments [1A] to [3A], wherein said composition comprises particles having an average particle size of from about 0.5 to about 15 μm.

Embodiment 5A. The dry powder composition of any one of Embodiments [1A] to [4A], wherein said composition has a density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

Embodiment 6A. The dry powder composition any one of Embodiments [1A] to [5A], wherein the relative weight ratio of pirfenidone to nintedanib or a pharmaceutically acceptable salt thereof in the dry powder composition is from about 8.0:1 to about 80:1, from about 8.0:1 to about 15:1; from about 8.5:1 to about 15:1; from about 9.0:1 to about 15:1, from about 10.0:1 to about 15:1, from about 11.0:1 to about 15:1, from about 12.0:1 to about 13.0:1, from about 13.0:1 to about 14.0:1.

Embodiment 7A. The dry powder composition of Embodiment [6A], wherein the relative weight ratio of pirfenidone to nintedanib or a pharmaceutically acceptable salt thereof in the dry powder is from about 9:1 to about 50:1.

Embodiment 8A. The dry powder composition of Embodiment [7A], wherein the relative weight ratio of pirfenidone to nintedanib in the dry powder is about 20:1.

Embodiment 9A. A dry powder composition suitable for direct pulmonary administration, which composition comprises:
an effective amount of pirfenidone and nintedanib or a pharmaceutically acceptable salt thereof;
an effective amount of di-stearoylphosphatidyl choline; and
an effective amount of calcium chloride;
   wherein said powder composition comprises particles having an average particle size of from about 0.5 to about 15 μm and a bulk density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

Embodiment 10A. The dry powder composition of Embodiment [9A], wherein pirfenidone and nintedanib or a pharmaceutically acceptable salt thereof are uniformly dispersed throughout in a ratio proportional to the amount of each used in the composition on a weight basis.

Embodiment 11A. The dry powder composition of Embodiment [10A], wherein the relative weight ratio of pirfenidone to nintedanib or a pharmaceutically acceptable salt thereof in the dry powder is from about 9:1 to about 50:1.

Embodiment 12A. The dry powder composition of Embodiment [11A], wherein the relative weight ratio of pirfenidone to nintedanib or a pharmaceutically acceptable salt of thereof in the dry powder is about 20:1.

Embodiment 13A. The dry powder composition of any one of Embodiments [9A] to [12A], wherein said composition further comprises one or more pharmaceutically acceptable additives.

Embodiment 14A. The dry powder composition of Embodiment [13A], wherein said one or more additives are selected from one or more dispersing agents, a buffering agent, a disaccharide, an antioxidant, a stabilizer, and mixtures of two or more additives.

Embodiment 15A. The dry powder composition of Embodiment [14A], wherein said dispersing agent is selected from leucine or trileucine or a combination of leucine and trileucine; and each or the combination optionally in the presence of a further dispersing agent.

Embodiment 16A. The dry powder composition of Embodiment [15A], wherein said further dispersing agent is polyethylene glycol sorbitan monooleate.

Embodiment 17A. The dry powder composition of Embodiment [14A], wherein the additive is a biocompatible buffer that is an acetate, citrate, phosphate, tartrate, and/or triethanolamine buffer.

Embodiment 18A. The dry powder composition of Embodiment [14A], wherein the additive is a disaccharide, and the disaccharide is trehalose, lactose (anhydrate or monohydrate), or mannitol.

Embodiment 19A. The dry powder composition of Embodiment [14A], wherein said antioxidant is selected from ascorbic acid, butylated hydroxyanisole, butylated hydroxytoluene, cysteine, glutathione, propyl gallate, sodium metabisulfite, sodium thiosulfate, and vitamin E.

Embodiment 20A. The dry powder composition of any one of Embodiments [9A] to [19A], wherein said pharmaceutically acceptable salt of nintedanib is nintedanib esylate.

Embodiment 21A. A method for treating a patient diagnosed with pulmonary fibrosis (PF), which method comprises:
a) administering to the lungs of the patient a dry powder composition which itself comprises particles comprising:
i) an effective amount of a pharmaceutical combination of pirfenidone and optionally an effective amount of treprostinil, treprostinil palmitil, or treprostinil sodium;
ii) nintedanib or a pharmaceutically acceptable salt thereof;
wherein the particles further comprise a di-fatty acid phosphatidyl choline and calcium chloride in an amount sufficient to impart structural stability;
and further wherein said particles in said dry powder composition have an average particle size of from about 0.5 to about 15 μm and the dry powder composition has a bulk density of from about 0.03 g/cc to about 0.30 g/cc, and the PF is as interstitial lung disease, progressive fibrosing interstitial lung disease, systemic sclerosis-associated interstitial lung disease, or idiopathic pulmonary fibrosis and pulmonary fibrosis.

Embodiment 22A. The method of Embodiment [21A], wherein the PF is classified as idiopathic pulmonary fibrosis or pulmonary fibrosis.

Embodiment 23A. The method of any one of Embodiments [21A] or [22A], wherein said pharmaceutically acceptable salt of nintedanib is nintedanib esylate.

Embodiment 24A. A method for treating a patient diagnosed with pulmonary fibrosis, which method comprises:
administering to the lungs of the patient an effective amount of the dry powder composition of any one of Embodiments [1A] to [20A], wherein the step of administering to the patient is a sequential administration in any order of pirfenidone and nintedanib or a pharmaceutically acceptable salt or a contemporaneous administration of pirfenidone and nintedanib or a pharmaceutically acceptable salt,
wherein said dry powder composition comprises particles having an average particle size of from about 0.5 to about 15 μm; the powder composition obtained by spray drying has a bulk density of from about 0.03 g/cc to about 0.30 g/cc, and wherein the PF is idiopathic pulmonary fibrosis (IPD), interstitial lung disease, progressive fibrosing interstitial lung disease, or systemic sclerosis-associated interstitial lung disease.

Embodiment 25A. The method of any one of Embodiments [21A] to [24A], wherein the patient is qualified as having stage 1 or stage 2 PF.

Embodiment 26A. The method of any one of Embodiments [21A] to [24A], wherein the patient qualifies as having incipient or moderately developed idiopathic pulmonary fibrosis but is not diagnosed with hypoxic respiratory failure or resting hypoxemia.

Embodiment 27A. The method of any one of Embodiments [21A] to [24A], wherein said method reduces disease progression in the patient.

Embodiment 28A. The method of any one of Embodiments [21A] to [24A], wherein the method reduces or prevents in the treated patient at least one symptom and/or at least one adverse event associated with an oral administration of pirfenidone, an oral administration of nintedanib or a pharmaceutically acceptable salt thereof, or an oral administration of both pirfenidone and nintedanib or a pharmaceutically acceptable salt of nintedanib.

Embodiment 29A. The method of Embodiment [28A], wherein the at least one symptom and/or the at least one adverse event with oral administration from pirfenidone administration selected from the group consisting of: diarrhea, nausea, vomiting, a skin ulcer, abdominal pain, an elevated liver enzyme level, patient weight decrease, fatigue, decreased patient appetite, a headache, pyrexia, back pain, dizziness, and hypertension, and
the at least one adverse event from administration of nintedanib or a pharmaceutically acceptable salt of nintedanib selected from the group consisting of: nausea, a rash, stomach pain, an upper respiratory tract infection, diarrhea, fatigue, a headache, indigestion, dizziness, decreased patient appetite or loss of appetite, gastroesophageal reflux disease (GERD), sinusitis, sleeplessness, weight loss, and joint pain.

Embodiment 30A. The method of any one of Embodiments [21A] to [26A], wherein said pharmaceutically acceptable salt of nintedanib is nintedanib esylate.

Embodiment 31A. The method of Embodiment [29A], wherein the elevated liver enzyme is aspartate transaminase, alanine transaminase, or gamma-glutamyl transpeptidase.

Embodiment 32A. The method of any one of Embodiments [21A] to [24A], wherein the step of administering is continued for the patient if one or more of the symptoms of pulmonary fibrosis have been reduced.

Embodiment 33A. The method of Embodiment [31A], wherein the symptoms of the patient are reduced compared to untreated patients.

Embodiment 34A. The method of Embodiment [32A], wherein the symptoms are selected from the group consisting: reduced coughing, improved carbon monoxide diffusion capacity, stabilization of dyspnea, and reduced periods of shortness of breath.

Embodiment 35A. The method of any one of Embodiments [21A] to [24A], wherein the patient with PF has a forced vital capacity (FVC) of more than about 40%±5% and/or a DLCO (lung diffusion capacity for carbon monoxide) of more than about 30%±5% predicted.

Embodiment 36A. The method of any one of Embodiments [21A] to [24A], wherein the effective amount of the pharmaceutical combination reduces the rate of disease progression in said patient by at least about 10%.

Embodiment 37A. The method of any one of Embodiments [21A] to [24A], wherein the effective amount of the pharmaceutical combination reduces the rate of disease progression in said patient by at least about 20%.

Embodiment 38A. The method of any one of Embodiments [21A] to [24A], wherein the effective amount of the pharmaceutical combination reduces the rate of disease progression in said patient by at least about 30%.

Embodiment 39A. The method of any one of Embodiments [21A] to [24A], wherein the effective amount of the pharmaceutical combination reduces the rate of disease progression in said patient by at least about 50%.

Embodiment 40A. The method of any one of Embodiments [21A] to [24A], wherein the effective amount of the pharmaceutical combination reduces the rate of disease progression in said patient by at least about 75%.

Embodiment 41A. A method for qualifying a patient for pharmacotherapy treatment of pulmonary fibrosis (PF) with the dry powder composition of any one of Embodiments [1A] to [20A], which method comprises:
evaluating said patient to determine the stage of pulmonary fibrosis for that patient;
initiating pharmacotherapy in a patient who is qualified as having stage 1 or stage 2 PF or disqualifying a patient from said pharmacotherapy if said patient is diagnosed as having stage 3 or stage 4 PF;
monitoring said qualified patient to determine the extent of progression of said disease after initiating pharmacotherapy; and
optionally removing said patient from pharmacotherapy for whom the attending clinician determines that the pharmacotherapy is no longer effective.

Embodiment 42. A method for treating qualified patients diagnosed with pulmonary fibrosis (PF) which method comprises:
initiating pharmacotherapy comprising administering the dry powder of any one of Embodiments [1A] to [20A] in a therapeutic amount to the patient qualified as having stage 1 or stage 2 PF;
maintaining said pharmacotherapy until the attending clinician determines that the disease progression in the patient has evolved such that there is no longer a benefit to the patient;
and terminating said pharmacotherapy.

Embodiment 43A. A method of making a dry powder composition comprising pirfenidone and nintedanib or a pharmaceutically acceptable salt thereof comprising the steps of:
preparing a nanoemulsion comprising perfluorooctyl bromide (PFOB) droplets, wherein the emulsion is stabilized by a plurality of DSPC liposomes in water,
admixing pirfenidone and nintedanib or a pharmaceutically acceptable salt thereof and the water present in a water phase in the nanoemulsion at a temperature of between about 40° C. to about 65° C.±5.0° C., wherein the nanoemulsion comprises 90% water, and
spray drying the nanoemulsion to obtain the powder composition.

Embodiment 44A. The method of Embodiment [43A], further comprising adjusting the pH of the admixed nanoemulsion to about 5.3=0.3 and wherein nintedanib or its pharmaceutically acceptable salt thereof is admixed in the emulsion first until dissolved, followed by admixing pirfenidone, which is further admixed until dissolved.

Embodiment 45A. The method of either Embodiment [43A] or Embodiment [44A], wherein said pharmaceutically acceptable salt of nintedanib is nintedanib esylate.

Embodiment 1B. A dry powder composition comprising an effective amount of a) pirfenidone, and a second medicament selected from b) nintedanib, treprostinil, treprostinil palmitil, or seralutinib, or a pharmaceutically acceptable salt thereof, and which further comprises c) a lecithin component.

Embodiment 2B. The dry powder composition of Embodiment [1B], wherein pirfenidone is deupirfenidone.

Embodiment 3B. The dry powder composition of Embodiment [1B], wherein the lecithin component is a di-fatty acid phosphatidyl choline.

Embodiment 4B. The dry powder composition of Embodiment [1B], further comprising a magnesium or calcium cation.

Embodiment 5B. The dry powder composition of Embodiment [1B], wherein said second medicament is nintedanib free base or a pharmaceutically acceptable salt of nintedanib.

Embodiment 6B. The dry powder composition of Embodiment [5B], wherein said pharmaceutically acceptable salt of nintedanib is an esylate salt.

Embodiment 7B. The dry powder composition of Embodiment [1B], wherein said second medicament is treprostinil or treprostinil palmitil.

Embodiment 8B. The dry powder composition of Embodiment [1B], wherein pirfenidone and said second medicament are uniformly dispersed in the dry powder composition.

Embodiment 9B. The dry powder composition of Embodiment [8B], wherein said composition comprises particles having an average particle size of from about 0.5 to about 15 μm.

Embodiment 10B. The dry powder composition of Embodiment [9B], wherein said composition has a density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

Embodiment 11B. The dry powder composition of Embodiment [10B], wherein a relative weight ratio of pirfenidone to nintedanib or a pharmaceutically acceptable salt thereof in the dry powder composition is from about 10.0:1 to about 40:1, from 15.0:1 to about 30.0:1, from about 19:1 to about 29:1, from about 20:1 to about 27:1, from about 24:1 to about 26:1, or from about 24:1 to about 25:1.

Embodiment 12B. The dry powder composition of Embodiment [11B], wherein the relative weight ratio of pirfenidone to nintedanib in the dry powder is about 25:1.

Embodiment 13B. A dry powder composition suitable for direct pulmonary administration, which composition comprises:
an effective amount of pirfenidone and nintedanib or a pharmaceutically acceptable salt thereof;
an effective amount of di-stearoylphosphatidyl choline;
an effective amount of calcium chloride; and
optionally one or more pharmaceutically acceptable additives,
wherein said powder composition comprises particles having an average particle size of from about 0.5 to about 15 μm and a bulk density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

Embodiment 14B. The dry powder composition of Embodiment [13B], wherein pirfenidone and nintedanib or a pharmaceutically acceptable salt thereof are uniformly dispersed throughout in a ratio proportional to an amount of each used in the composition on a weight basis.

Embodiment 15B. The dry powder composition of Embodiment [14B], wherein a relative weight ratio of pirfenidone to nintedanib or a pharmaceutically acceptable salt thereof in the dry powder is from about 10:1 to about 40:1 or about 24:1 to 26:1.

Embodiment 16B. The dry powder composition of Embodiment [13B], wherein the dry powder composition further comprises one or more pharmaceutically acceptable additives, and said pharmaceutically acceptable additives are:
a) one or more dispersing agents, wherein the dispersing agent is leucine, trileucine, and a combination of leucine and trileucine, and optionally comprising a further dispersing agent;
b) a biocompatible buffer selected from an acetate, citrate, histidine phosphate, tartrate, and/or triethanolamine buffer;
c) a disaccharide, and the disaccharide is trehalose or lactose (anhydrate or monohydrate); and/or
d) an antioxidant and the antioxidant is ascorbic acid, butylated hydroxyanisole, butylated hydroxytoluene, cysteine, glutathione, propyl gallate, sodium metabisulfite, sodium thiosulfate, methionine, or vitamin E.

Embodiment 17B. The dry powder composition of Embodiment [13B], wherein said pharmaceutically acceptable salt of nintedanib is nintedanib esylate.

Embodiment 18B. A method for treating a patient diagnosed with pulmonary fibrosis (PF), which method comprises:
a) administering to the patient via inhalation to a patient's lung or lungs a dry powder composition which itself comprises particles comprising:
i) an effective amount of a pharmaceutical combination of pirfenidone and optionally an effective amount of treprostinil, treprostinil palmitil, or treprostinil sodium; and
ii) nintedanib or a pharmaceutically acceptable salt thereof;
wherein the particles further comprise a di-fatty acid phosphatidyl choline and calcium chloride in an amount sufficient to impart structural stability;
and further wherein said particles in said dry powder composition have an average particle size of from about 0.5 to about 15 μm and the dry powder composition has a bulk density of from about 0.03 g/cc to about 0.30 g/cc, and the patient is diagnosed as having an interstitial lung disease, progressive fibrosing interstitial lung disease (PH-ILD), systemic sclerosis-associated interstitial lung disease (SSc-ILD), or idiopathic pulmonary fibrosis and pulmonary fibrosis.

Embodiment 19B. The method of Embodiment [18B], wherein the patient having PF is diagnosed with idiopathic pulmonary fibrosis or pulmonary fibrosis.

Embodiment 20B. The method of Embodiment [18B], wherein said pharmaceutically acceptable salt of nintedanib is nintedanib esylate.

Embodiment 21B. A method for treating a patient diagnosed with pulmonary fibrosis (PF), which method comprises:
administering to the patient's lung or lungs an effective amount of the dry powder composition of Embodiment [1B], wherein the step of administering to the patient is a sequential administration in any order, a fixed dose of pirfenidone and nintedanib or a pharmaceutically acceptable salt thereof, or a contemporaneous administration of a combination comprising a fixed dose of pirfenidone and nintedanib or a pharmaceutically acceptable salt thereof,
wherein said dry powder composition comprises particles having an average particle size of from about 0.5 to about 15 μm; the powder composition obtained by spray drying has a bulk density of from about 0.03 g/cc to about 0.30 g/cc, and wherein the PF is idiopathic pulmonary fibrosis (IPF), interstitial lung disease, progressive fibrosing interstitial lung disease (PF-ILD), or systemic sclerosis-associated interstitial lung disease.

Embodiment 22B. The method of Embodiment [21B], wherein the patient is qualified as having:
a) Stage 1 or Stage 2 PF, or
b) incipient or moderately developed idiopathic pulmonary fibrosis, but is not diagnosed with hypoxic respiratory failure or resting hypoxemia.

Embodiment 23B. The method of Embodiment [21B], wherein said method:
a) reduces disease progression in the patient,
b) reduces or prevents in the treated patient at least one symptom and/or at least one adverse event associated with an oral administration of pirfenidone, an oral administration of nintedanib or a pharmaceutically acceptable salt thereof, or an oral administration of both pirfenidone and nintedanib or a pharmaceutically acceptable salt of nintedanib,
c) reduces or prevents in the treated patient at least one symptom and/or at least one adverse event associated with an oral administration of pirfenidone, an oral administration of nintedanib or a pharmaceutically acceptable salt thereof, or an oral administration of both pirfenidone and nintedanib or a pharmaceutically acceptable salt of nintedanib, or
d) reduces or prevents in the treated patient at least one symptom and/or at least one adverse event associated with an oral administration of pirfenidone or a pharmaceutically acceptable salt thereof, or an oral administration of both pirfenidone and nintedanib or salts thereof, wherein
(i) the at least one symptom and/or at least one adverse event from pirfenidone administration is diarrhea, nausea, vomiting, a skin ulcer, abdominal pain, an elevated liver enzyme level, patient weight decrease, fatigue, decreased patient appetite, a headache, pyrexia, back pain, dizziness, or hypertension, and
(ii) the at least one symptom and/or at least one adverse event nintedanib administration or a pharmaceutically acceptable salt of nintedanib is: nausea, a rash, stomach pain, an upper respiratory tract infection, diarrhea, fatigue, a headache, indigestion, dizziness, decreased patient appetite or loss of appetite, gastroesophageal reflux disease (GERD), sinusitis, sleeplessness, weight loss, or joint pain.

Embodiment 24B. The method of Embodiment [21B], wherein the step of administering is continued for the patient if one or more of the symptoms of pulmonary fibrosis have been reduced, wherein the one or more symptoms of pulmonary fibrosis are selected from a group consisting of: reduced coughing, improved carbon monoxide diffusion capacity, stabilization of dyspnea, and reduced periods of shortness of breath.

Embodiment 25B. The method of Embodiment [21B], wherein the patient with PF has a forced vital capacity (FVC) of more than about 40%±5% and/or a DLCO (lung diffusion capacity for carbon monoxide) of more than about 30%±5%.

Embodiment 26B. The method of Embodiment [21B], wherein the effective amount of the pharmaceutical combination reduces a PF progression rate in said patient by at least about 10%, at least 20%, at least 30% or by at least 50%, and/or improves patient-reported outcomes (e.g., improved 6-minute walk distances).

Embodiment 27B. A method for qualifying a patient for pharmacotherapy treatment of pulmonary fibrosis (PF) with the dry powder composition of Embodiment [1B], which method comprises:
a) evaluating said patient to determine a pulmonary fibrosis stage for the patient, wherein the evaluated patient is qualified as having Stage 1 or Stage 2 PF but not Stage 3, or Stage 4 PF;
b) initiating pharmacotherapy in the patient or disqualifying a patient from said pharmacotherapy if said patient is diagnosed as having Stage 3 or Stage 4 PF;
c) monitoring said qualified patient to determine the extent of progression of PF after initiating pharmacotherapy; and
optionally removing said patient from pharmacotherapy for whom the attending clinician determines that the pharmacotherapy is no longer effective.

Embodiment 28B. A method for treating a patient qualified as having pulmonary fibrosis (PF), which method comprises:
initiating pharmacotherapy comprising administering the dry powder of

Embodiment [1B] in a therapeutic amount to the qualified patient, wherein the patient is qualified as having Stage 1 or Stage 2 PF;
maintaining said pharmacotherapy until an attending clinician determines that the patient no longer benefits from the pharmacotherapy due to PF progression; and
terminating said pharmacotherapy.

Embodiment 29B. A method of preparing a dry powder composition comprising pirfenidone and nintedanib or a pharmaceutically acceptable salt thereof, the method comprising:
a) emulsifying a suspension medium comprising pirfenidone, nintedanib or a pharmaceutically acceptable salt thereof, and a di-fatty acid phosphatidyl choline in a volatile solvent to form a stable nanoemulsion;
b) atomizing the stable nanoemulsion into droplets; and
c) spraying the droplets into a drying chamber, wherein heated drying gas removes the volatile solvent, thereby obtaining the dry powder composition,
wherein an outlet temperature is controlled at no more than 75° C.

Embodiment 30B. The method of Embodiment [29B], wherein the suspension medium further comprises calcium chloride or magnesium chloride.

Embodiment 31B. The method of Embodiment [29B], wherein said pharmaceutically acceptable salt of nintedanib is nintedanib esylate.

Embodiment 32B. The dry powder composition prepared by the method of Embodiment [29B].

Embodiment 33B. A composition comprising the dry powder composition of Embodiment [1B], wherein the dry powder composition comprises particles comprising:
a) an effective amount of a pharmaceutical combination of i) pirfenidone, ii) treprostinil palmitil, and iii) optionally nintedanib, that can be administered by oral inhalation;
b) a di-fatty acid phosphatidyl choline compound in an amount sufficient to impart structural stability to said particles; and
c) an effective amount of a pharmaceutically acceptable magnesium salt or a calcium salt;
wherein the dry powder composition comprises particles having an average particle size (diameter along a longest axis) of from about 0.5 to about 15 μm or about 2 to about 6 μm; a bulk density of from about 0.03 g/cc to about 0.30 g/cc, or about 0.04 g/cc to about 0.11 g/cc.

Embodiment 34B. A method for treating a patient with pulmonary fibrosis (PF), pulmonary arterial hypertension (PAH), or pulmonary hypertension in interstitial lung disease (PH-ILD), the method comprising:
a) confirming that the patient is currently diagnosed with pulmonary fibrosis, pulmonary arterial hypertension (PAH), or pulmonary hypertension in interstitial lung disease (PH-ILD); and
b) administering directly to a patient's lung or lungs the composition of Embodiment [33B].

Embodiment 35B. The method of Embodiment [34B], wherein said administration is conducted twice daily.

Embodiment 36B. A dry powder composition comprising an effective amount of:
a) a first medicament which is nintedanib, and
b) optionally a second medicament selected from treprostinil, treprostinil palmitil, seralutinib, or pirfenidone, or a pharmaceutically acceptable salt thereof, and which further comprises a lecithin component, optionally a di-fatty acid phosphatidyl choline.

Embodiment 37B. The dry powder composition of Embodiment [36B], wherein the first medicament is nintedanib having methyl as R group with the proviso that the dry powder composition does not contain lactose (optionally lactose monohydrate) or fumaryl diketopiperazine (FDKP) as a carrier, or
wherein the first medicament is a longer chain ester form of nintedanib having-C₈₋₂₀ alkyl as R group, optionally having —C₁₆ alkyl as R group.

Embodiment 38B. A method for treating a patient with pulmonary fibrosis (PF), pulmonary arterial hypertension (PAH), or pulmonary hypertension in interstitial lung disease (PH-ILD), the method comprising:
a) confirming that the patient is currently diagnosed with pulmonary fibrosis, pulmonary arterial hypertension (PAH), or pulmonary hypertension in interstitial lung disease (PH-ILD); and
b) administering directly to a patient's lung or lungs the dry powder composition of Embodiment [36B].

Embodiment 39B. The method of Embodiment [38B], wherein said administration is conducted once daily.

What is claimed is:

1. A dry powder composition comprising an effective amount of a) pirfenidone, and a second medicament selected from b) nintedanib, treprostinil, treprostinil palmitil, or seralutinib, or a pharmaceutically acceptable salt thereof, and which further comprises c) a lecithin component.

2. The dry powder composition of claim 1, wherein pirfenidone is deupirfenidone.

3. The dry powder composition of claim 1, wherein the lecithin component is a di-fatty acid phosphatidyl choline.

4. The dry powder composition of claim 1, further comprising a magnesium or calcium cation.

5. The dry powder composition of claim 1, wherein said second medicament is nintedanib free base or a pharmaceutically acceptable salt of nintedanib.

6. The dry powder composition of claim 5, wherein said pharmaceutically acceptable salt of nintedanib is an esylate salt.

7. The dry powder composition of claim 1, wherein said second medicament is treprostinil or treprostinil palmitil.

8. The dry powder composition of claim 1, wherein pirfenidone and said second medicament are uniformly dispersed in the dry powder composition.

9. The dry powder composition of claim 8, wherein said composition comprises particles having an average particle size of from about 0.5 to about 15 μm.

10. The dry powder composition of claim 9, wherein said composition has a density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

11. The dry powder composition of claim 10, wherein a relative weight ratio of pirfenidone to nintedanib or a pharmaceutically acceptable salt thereof in the dry powder composition is from about 10.0:1 to about 40:1, from 15.0:1 to about 30.0:1, from about 19:1 to about 29:1, from about 20:1 to about 27:1, from about 24:1 to about 26:1, or from about 24:1 to about 25:1.

12. The dry powder composition of claim 11, wherein the relative weight ratio of pirfenidone to nintedanib in the dry powder is about 25:1.

13. A dry powder composition suitable for direct pulmonary administration, which composition comprises:
a) an effective amount of pirfenidone and nintedanib or a pharmaceutically acceptable salt thereof;
b) an effective amount of di-stearoylphosphatidyl choline;
c) an effective amount of calcium chloride; and
d) optionally one or more pharmaceutically acceptable additives,
wherein said powder composition comprises particles having an average particle size of from about 0.5 to about 15 μm and a bulk density of from about 0.03 g/cc to about 0.30 g/cc, from about 0.04 g/cc to about 0.10 g/cc, from about 0.05 g/cc to about 0.09 g/cc, or from about 0.06 g/cc to about 0.08 g/cc.

14. The dry powder composition of claim 13, wherein pirfenidone and nintedanib or a pharmaceutically acceptable salt thereof are uniformly dispersed throughout in a ratio proportional to an amount of each used in the composition on a weight basis.

15. The dry powder composition of claim 14, wherein a relative weight ratio of pirfenidone to nintedanib or a pharmaceutically acceptable salt thereof in the dry powder is from about 10:1 to about 40:1 or about 24:1 to 26:1.

16. The dry powder composition of claim 13, wherein the dry powder composition further comprises one or more pharmaceutically acceptable additives, and said pharmaceutically acceptable additives are:
a) one or more dispersing agents, wherein the dispersing agent is leucine, trileucine, and a combination of leucine and trileucine, and optionally comprising a further dispersing agent;
b) a biocompatible buffer selected from an acetate, citrate, histidine phosphate, tartrate, and/or triethanolamine buffer;
c) a disaccharide, and the disaccharide is trehalose or lactose (anhydrate or monohydrate);
d) an antioxidant and the antioxidant is ascorbic acid, butylated hydroxyanisole, butylated hydroxytoluene, cysteine, glutathione, propyl gallate, sodium metabisulfite, sodium thiosulfate, methionine, or vitamin E.

17. The dry powder composition of claim 13, wherein said pharmaceutically acceptable salt of nintedanib is nintedanib esylate.

18. A method for treating a patient diagnosed with pulmonary fibrosis (PF), which method comprises:
a) administering to the patient via inhalation to a patient's lung or lungs a dry powder composition which itself comprises particles comprising:
i) an effective amount of a pharmaceutical combination of pirfenidone and optionally an effective amount of treprostinil, treprostinil palmitil, or treprostinil sodium; and
ii) nintedanib or a pharmaceutically acceptable salt thereof;
wherein the particles further comprise a di-fatty acid phosphatidyl choline and calcium chloride in an amount sufficient to impart structural stability;
and further wherein said particles in said dry powder composition have an average particle size of from about 0.5 to about 15 μm and the dry powder composition has a bulk density of from about 0.03 g/cc to about 0.30 g/cc, and the patient is diagnosed as having an interstitial lung disease, progressive fibrosing interstitial lung disease (PH-ILD), systemic sclerosis-associated interstitial lung disease (SSc-ILD), or idiopathic pulmonary fibrosis and pulmonary fibrosis.

19. The method of claim 18, wherein the patient having PF is diagnosed with idiopathic pulmonary fibrosis or pulmonary fibrosis.

20. The method of claim 18, wherein said pharmaceutically acceptable salt of nintedanib is nintedanib esylate.

21. A method for treating a patient diagnosed with pulmonary fibrosis (PF), which method comprises:
administering to the patient's lung or lungs an effective amount of the dry powder composition of claim 1, wherein the step of administering to the patient is a sequential administration in any order, a fixed dose of pirfenidone and nintedanib or a pharmaceutically acceptable salt thereof, or a contemporaneous administration of a combination comprising a fixed dose of pirfenidone and nintedanib or a pharmaceutically acceptable salt thereof,
wherein said dry powder composition comprises particles having an average particle size of from about 0.5 to about 15 μm; the powder composition obtained by spray drying has a bulk density of from about 0.03 g/cc to about 0.30 g/cc, and wherein the PF is idiopathic pulmonary fibrosis (IPF), interstitial lung disease, progressive fibrosing interstitial lung disease (PF-ILD), or systemic sclerosis-associated interstitial lung disease.

22. The method of claim 21, wherein the patient is qualified as having:
a) Stage 1 or Stage 2 PF, or
b) incipient or moderately developed idiopathic pulmonary fibrosis, but is not diagnosed with hypoxic respiratory failure or resting hypoxemia.

23. The method of claim 21, wherein said method:
a) reduces disease progression in the patient,
b) reduces or prevents in the treated patient at least one symptom and/or at least one adverse event associated with an oral administration of pirfenidone, an oral administration of nintedanib or a pharmaceutically acceptable salt thereof, or an oral administration of both pirfenidone and nintedanib or a pharmaceutically acceptable salt of nintedanib,
c) reduces or prevents in the treated patient at least one symptom and/or at least one adverse event associated with an oral administration of pirfenidone, an oral administration of nintedanib or a pharmaceutically acceptable salt thereof, or an oral administration of both pirfenidone and nintedanib or a pharmaceutically acceptable salt of nintedanib, or
d) reduces or prevents in the treated patient at least one symptom and/or at least one adverse event associated with an oral administration of pirfenidone or a pharmaceutically acceptable salt thereof, or an oral administration of both pirfenidone and nintedanib or salts thereof, wherein
(i) the at least one symptom and/or at least one adverse event from pirfenidone administration is diarrhea, nausea, vomiting, a skin ulcer, abdominal pain, an elevated liver enzyme level, patient weight decrease, fatigue, decreased patient appetite, a headache, pyrexia, back pain, dizziness, or hypertension, and
(ii) the at least one symptom and/or at least one adverse event nintedanib administration or a pharmaceutically acceptable salt of nintedanib is: nausea, a rash, stomach pain, an upper respiratory tract infection, diarrhea, fatigue, a headache, indigestion, dizziness, decreased patient appetite or loss of appetite, gastroesophageal reflux disease (GERD), sinusitis, sleeplessness, weight loss, or joint pain.

24. The method of claim 21, wherein the step of administering is continued for the patient if one or more of the symptoms of pulmonary fibrosis have been reduced, wherein the one or more symptoms of pulmonary fibrosis are selected from a group consisting of: reduced coughing, improved carbon monoxide diffusion capacity, stabilization of dyspnea, and reduced periods of shortness of breath.

25. The method of claim 21, wherein the patient with PF has a forced vital capacity (FVC) of more than about 40%±5% and/or a DLCO (lung diffusion capacity for carbon monoxide) of more than about 30%±5%.

26. The method of claim 21, wherein the effective amount of the pharmaceutical combination reduces a PF progression rate in said patient by at least about 10%, at least 20%, at least 30% or by at least 50%, and/or improves patient-reported outcomes (e.g., improved 6-minute walk distances).

27. A method for qualifying a patient for pharmacotherapy treatment of pulmonary fibrosis (PF) with the dry powder composition of claim 1, which method comprises:
a) evaluating said patient to determine a pulmonary fibrosis stage for the patient, wherein the evaluated patient is qualified as having Stage 1 or Stage 2 PF but not Stage 3, or Stage 4 PF;
b) initiating pharmacotherapy in the patient or disqualifying a patient from said pharmacotherapy if said patient is diagnosed as having Stage 3 or Stage 4 PF;
c) monitoring said qualified patient to determine the extent of progression of PF after initiating pharmacotherapy; and
d) optionally removing said patient from pharmacotherapy for whom the attending clinician determines that the pharmacotherapy is no longer effective.

28. A method for treating a patient qualified as having pulmonary fibrosis (PF), which method comprises:
a) initiating pharmacotherapy comprising administering the dry powder of claim 1 in a therapeutic amount to the qualified patient, wherein the patient is qualified as having Stage 1 or Stage 2 PF;
b) maintaining said pharmacotherapy until an attending clinician determines that the patient no longer benefits from the pharmacotherapy due to PF progression; and
c) terminating said pharmacotherapy.

29. A method of preparing a dry powder composition comprising pirfenidone and nintedanib or a pharmaceutically acceptable salt thereof, the method comprising:
a) emulsifying a suspension medium comprising pirfenidone, nintedanib or a pharmaceutically acceptable salt thereof, and a di-fatty acid phosphatidyl choline in a volatile solvent to form a stable nanoemulsion;
b) atomizing the stable nanoemulsion into droplets; and
c) spraying the droplets into a drying chamber, wherein heated drying gas removes the volatile solvent, thereby obtaining the dry powder composition,
wherein an outlet temperature is controlled at no more than 72° C.

30. The method of claim 29, wherein the suspension medium further comprises calcium chloride or magnesium chloride.

31. The method of claim 29, wherein said pharmaceutically acceptable salt of nintedanib is nintedanib esylate.

32. The dry powder composition prepared by the method of claim 29.

33. A composition comprising the dry powder composition of claim 1, wherein the dry powder composition comprises particles comprising:
a) an effective amount of a pharmaceutical combination of i) pirfenidone, ii) treprostinil palmitil, and iii) optionally nintedanib, that can be administered by oral inhalation;
b) a di-fatty acid phosphatidyl choline compound in an amount sufficient to impart structural stability to said particles; and
c) an effective amount of a pharmaceutically acceptable magnesium salt or a calcium salt;
wherein the dry powder composition comprises particles having an average particle size (diameter along a longest axis) of from about 0.5 to about 15 μm or about 2 to about 6 μm; a bulk density of from about 0.03 g/cc to about 0.30 g/cc, or about 0.04 g/cc to about 0.11 g/cc.

34. A method for treating a patient with pulmonary fibrosis (PF), pulmonary arterial hypertension (PAH), or pulmonary hypertension in interstitial lung disease (PH-ILD), the method comprising:
a) confirming that the patient is currently diagnosed with pulmonary fibrosis, pulmonary arterial hypertension (PAH), or pulmonary hypertension in interstitial lung disease (PH-ILD); and
b) administering directly to a patient's lung or lungs the composition of claim 33.

35. The method of claim 34, wherein said administration is conducted twice daily.

36. A dry powder composition comprising an effective amount of:
a) a first medicament which is nintedanib, and
b) optionally a second medicament selected from treprostinil, treprostinil palmitil, seralutinib, or pirfenidone, or a pharmaceutically acceptable salt thereof, and which further comprises a lecithin component, optionally a di-fatty acid phosphatidyl choline.

37. The dry powder composition of claim 36, wherein the first medicament is nintedanib having methyl as R group with the proviso that the dry powder composition does not contain lactose (optionally lactose monohydrate) or fumaryl diketopiperazine (FDKP) as a carrier, or
wherein the first medicament is a longer chain ester form of nintedanib having —C₈₋₂₀ alkyl as R group, optionally having —C₁₆ alkyl as R group.

38. A method for treating a patient with pulmonary fibrosis (PF), pulmonary arterial hypertension (PAH), or pulmonary hypertension in interstitial lung disease (PH-ILD), the method comprising:
a) confirming that the patient is currently diagnosed with pulmonary fibrosis, pulmonary arterial hypertension (PAH), or pulmonary hypertension in interstitial lung disease (PH-ILD); and
b) administering directly to a patient's lung or lungs the dry powder composition of claim 36.

39. The method of claim 38, wherein said administration is conducted once daily.
